(19) United States
(12) Patent Application Publication
(10) Pub. No.: US 2018/0028601 A1
(43) Pub. Date: Feb. 1, 2018

(54) METHODS AND COMPOSITIONS FOR TREATING CANCER

(71) Applicant: Yissum Research Development Company of The Hebrew University of Jerusalem Ltd., Jerusalem (IL)

(72) Inventors: Gilad Benjamin BACHRACH, Moshav Beit Zait (IL); Ofer MANDELBOIM, Shoham (IL); Jawad ABED, Nazareth (IL)

(73) Assignee: Yissum Research Development Company of The Hebrew University of Jerusalem Ltd., Jerusalem (IL)

(21) Appl. No.: 15/658,747

(22) Filed: Jul. 25, 2017

Related U.S. Application Data
(60) Provisional application No. 62/366,724, filed on Jul. 26, 2016.

(51) Int. Cl. A61K 38/16; C07K 16/12; A61K 49/00; A61K 45/06; A61K 39/395; G01N 33/574; C07K 16/44

(52) U.S. Cl. CPC A61K 38/164; G01N 33/57492; C07K 16/1203; C07K 16/44; A61K 45/06; A61K 39/39558; A61K 49/0002; G01N 2400/00; G01N 2333/195

(57) ABSTRACT

Provided are compositions and methods of targeting imaging or therapeutic agents to cancer sites using Fap2. Also provided are methods of treating cancer by inhibiting the complexing of Fusobacterium nucleatum Fap2 and Gal-GalNAc molecules expressed on cancer cells.

### FIELD OF THE INVENTION

The invention is in the field of cancer therapy and relates to compositions and methods of targeting imaging or therapeutic agents to cancer sites using Fap2. The present invention further relates to treating adenocarcinoma by inhibiting the binding of Fusobacterium nucleatum Fap2 to Gal-GalNAc molecules presented on cancer cells.

### BACKGROUND OF THE INVENTION

Colorectal cancer (CRC) is the third leading cause of cancer-related deaths in the United States and microbes have emerged as key factors that influence the development, progression, and response to treatment of CRC. For example, enterotoxigenic Bacteroides fragilis accelerates colon tumor development by inducing an acute and self-limited colitis triggering an Il-23 and Il-17 inflammatory response in intestinal adenoma-prone Apc^{Min/+} mice. Colibactin-expressing Escherichia coli potentiates colorectal carcinogenesis in azoxymethane-exposed gnotobiotic Il10^{-/-} mice. In addition, carbohydrate-derived bacterial metabolites, such as butyrate, can increase hyperproliferation in Msh2^{-/-} (DNA mismatch repair gene MutS homolog 2) colon epithelial cells; in contrast with ingestion of a low fiber diet that reduces tumor numbers in Apc^{Min/+}Msh2^{-/-} mice. These data reflect a spectrum of ways by which bacteria contribute to colorectal carcinogenesis.

Recent metagenomic and transcriptomic analyses have revealed an enrichment of Fusobacterium species in human colorectal cancers and adenomas compared to adjacent normal tissue (Castellarin et al. 2012, Genome Res. 2012. 22: 299-306; Kostic et al., 2012, Genome Res. 2012. 22: 292-298). Increased levels of F. nucleatum correlate with specific molecular subsets of colorectal cancers such as the CpG island methylator phenotype (CIMP) and microsatellite instability (MSI). F. nucleatum accelerates CRC in preclinical models using both in vitro and in vivo systems (Kostic et al., 2013. Cell Host Microbe. 2013; 14: 207-215; Rubinstein et al., 2013, Cell Host Microbe. 2013; 14: 195-206). F. nucleatum also suppresses anti-tumor immunity and inhibits tumor killing by natural killer (NK) cells and tumor-infiltrating lymphocytes (Gur et al., 2015, Immunity 42, 344-355). All of these findings support that F. nucleatum not only localizes to and is enriched in colon adenomas and colorectal adenocarcinoma but also may function in tumor growth and survival.

Once in the tumor, fusobacteria can accelerate cancer development by enhancing cellular proliferation, creating a tumor-favorable inflammatory environment and by protecting tumors from killing by NK cells and tumor infiltrating T cells. Not surprisingly, high fusobacterial abundance in CRC was correlated with poor disease outcome (Flanagan et al., 2014. Eur. J. Clin. Microbiol. Infect. Dis. 33, 1381-1390), suggesting that prevention or reduction of CRC-fusobacteria interactions should be considered therapeutically.

Fusobacterium nucleatum is a gram-negative anaerobe from the oral cavity that plays a key role in the development of the dental plaque by physically bridging between early and late oral bacterial colonizers. F. nucleatum numbers rise 10,000 fold in the gingival inflammation that precedes periodontal disease. F. nucleatum is also frequently isolated (often as pure cultures) from samples collected in preterm births.

WO 2012/045150 discloses methods for prognosing or diagnosing a gastrointestinal cancer in a subject comprising: providing a sample from the subject; and detecting a

Fusobacterium sp. in the sample, wherein a positive detection of the Fusobacterium sp. indicates a prognosis or diagnosis of gastrointestinal cancer.

Many different molecular and cellular mechanisms have been reported so far for cancer growth and metastasis. There is an unmet need to provide additional approaches with improved effectiveness for treating cancer, having fewer or no side effects.

### SUMMARY OF THE INVENTION

The present invention provides methods and compositions for treating cancer. The methods and compositions described herein are based on the unexpected discovery that Fap2, an outer membrane protein of Fusobacterium nucleatum, mediates binding of the bacteria to tumors that display Gal-GalNAc. The present invention in some embodiments provides methods for treating cancer that comprise specific targeting therapeutic agents, associated or covalently linked to Fap2, to tumor sites. Fap2 is employed as a binding moiety that directs molecules to tumors. The compositions of the invention in some embodiments, can reduce the binding of Fap2 protein, expressed on the fusobacterium, to Gal-GalNAc molecules present on tumors. The present invention in some embodiments further provides methods of diagnosing cancer that comprise applying Fap2 as a tumor targeting moiety. The present invention in additional embodiments provides methods for treating cancer that comprise administering an inhibitor of the interaction between Fap2 and Gal-GalNAc. The methods and compositions disclosed herein are useful in cancer therapy as stand-alone therapy and in combination with other anti-cancer agents.

It is now disclosed that Fap2/Gal-GalNAc interaction constitutes the main pathway for fusobacteria homing to tumor sites, thus inhibition of the interaction may be used for treating cancer. Fap2 binding to Gal-GalNAc makes it a valuable candidate for use in cancer therapy, enabling targeting of therapeutic agents to the cancer sites. Directing therapeutics to a cancer site enable administration of lower doses with fewer side effects.

The inventors of the present invention showed that Fap2 deficient bacteria failed to attach to tumor tissues. It is now disclosed that Fap2 mediates CRC colonization by F. nucleatum in the CT26 colorectal cancer model. It is further disclosed that a variety of adenocarcinomas were found to present high levels of Gal-GalNAc, hence the disaccharide embodies as a good and efficient target for cancer therapy.

According to one aspect, the present invention provides a composition comprising:
(i) a Fap2 protein or a fragment thereof comprising a Gal-GalNAc binding site; and
(ii) a therapeutic or diagnostic agent.

According to some embodiments, the therapeutic agent is an immunotherapeutic agent.

According to certain embodiments, the therapeutic agent is a chemotherapeutic agent.

According to some embodiments, the therapeutic agent is a polypeptide capable of inducing cell death in the cancer cell. According to additional embodiments, the composition comprises Fap2 expressed by bacterium.

According to some embodiments, the composition comprises a modified Fusobactrium expressing Fap2. According to certain embodiments, the modified Fusobactrium comprises a toxin.

According to certain embodiments, the Fusobacterium is engineered not to bind or activate TIGIT.

According to some embodiments, the Fap2 is a mutated protein.

According to certain embodiments, the Fap2 is engineered not to bind or activate TIGIT.

According to some embodiments, Fap2 is directly coupled to the therapeutic or diagnostic agent. According to some embodiments, Fap2 is coupled to the therapeutic or diagnostic agent through a linker.

According to other embodiments, Fap2 is associated with the therapeutic or diagnostic agent.

According to certain embodiments, the Fap2 is conjugated to a delivery agent.

According to certain embodiments, the delivery agent is a micro or nanoparticle.

According to some embodiments, the composition is a liposome comprising the therapeutic or diagnostic agent in its core and/or lipid membraned. According to certain embodiments, the liposome presents at least one Fap2 molecule on its membrane.

According to some embodiments, the therapeutic agent or the diagnostic agent comprises a radioisotope or a photoactive agent.

According to some embodiments, the therapeutic or diagnostic agent is a quantum dot.

According to some embodiments, the diagnostic agent is selected from the group consisting of: fluorescent agent, radio-imaging agent, photo-imaging agent, and an agent used to perform or enhance CT, MRI, or ultrasound imaging. Each possibility represents a separate embodiment of the invention.

According to some embodiments, the composition is a pharmaceutical composition further comprising an acceptable pharmaceutical carrier.

According to some embodiments, the pharmaceutical composition is formulated for parenteral administration. For example, the pharmaceutical compositions may be formulated for injection administration, including but not limited to intravenous, intra-articular, intramuscular, subcutaneous, intradermal or intrathecal. Each possibility represents a separate embodiment of the present invention.

According to other embodiments, the pharmaceutical composition is formulated for local administration. For example, the pharmaceutical compositions may be formulated for direct administration to the treated body site or tissue. According to some embodiments, the pharmaceutical composition is formulated for direct administration to the rectum.

According to an aspect, the present invention provides a method of treating cancer characterized by elevated amounts of Gal-GalNAc in a subject, the method comprising administering to the subject a therapeutically effective amount of the pharmaceutical composition as described hereinabove.

According to some embodiments, the cancer cell is adenocarcinoma. According to certain embodiments, the adenocarcinoma is selected from the group consisting of: colon cancer, ovarian cancer, stomach cancer, uterus, cervical cancer, breast cancer, endometrial cancer, prostate cancer, lung cancer, and pancreatic cancer. According to some embodiments, the cancer is colorectal carcinoma. According to some embodiment, the method further comprising treating with an additional anticancer therapy.

According to some embodiments, the Gal-GalNAc molecules amount is elevated in cancer cells by at least 10%, 20%, 30%, 40%, 50%, 60%, 70%, 80%, 90%, or 100% compared to a normal cell. Each possibility represents a separate embodiment of the invention. According to some embodiments, the pharmaceutical composition is administered via a route of administration selected from the group consisting of: intravenously, subcutaneously, intra-arterially, intraperitoneally, intramuscularly, rectally, vaginally, intradermally, intraventricularly, intracisternally, intracapsularly, intrapulmonarily, intranasally, transmucosally, transdermally, inhalation, and any combination thereof. Each possibility represents a separate embodiment of the invention. According to certain embodiments, the composition is administered intravenously.

According to other embodiments, the pharmaceutical composition is administered locally or directly to the treated body site or tissue. According to some embodiments, the pharmaceutical composition is administered directly to the rectum. According to some embodiments the pharmaceutical composition is administered locally or directly during surgery to the treated or removed tumor tissue.

According to some embodiments, the subject is human.

According to some embodiments, the composition further reduces the binding of Fap2 protein expressed on the fusobacteria and Gal-GalNAc molecules present on tumor cells.

According to an additional aspect, the present invention provides a method of diagnosing a cancer in a subject, said cancer is characterized by elevated amounts of Gal-GalNAc molecules on cell surface, the method comprising administering to the subject the composition comprising the diagnostic agent as described hereinabove.

According to an additional aspect, the present invention provides a method of diagnosing a cancer in a subject, said cancer is characterized by elevated amounts of Gal-GalNAc molecules on cell surface, the method comprising determining the expression level of Gal-GalNAc in a biological sample of said subject using the composition as described herein.

According to some embodiments, the method is ex-vivo or in-vitro.

According to some embodiments, the Fap2 is covalently linked to an imaging agent. According to some embodiments, the Fap2 binding to Gal-GalNAc is determined by an antibody specific to Fap2.

According to some embodiments, the method further comprises comparing the expression level of Gal-GalNAc with a control or a reference sample.

According to another aspect, the present invention provides a method of determining or quantifying the Gal-GalNAc amounts, the method comprising contacting a biological sample with Fap2 protein or Fap2 fragment, and measuring the level of complex formation.

Determining and quantifying methods may be performed in-vitro or ex-vivo according to some embodiments or may be used in diagnosing conditions or diseases associated with overexpression or over presenting of Gal-GalNAc.

According to another aspect the present invention provides a method of treating cancer, the method comprising administering to a subject in need thereof a therapeutically effective amount of at least one agent that reduces the binding of Fap2 to Gal-GalNAc present on cancer cells.

According to some embodiments, the cancer is adenocarcinoma. According to certain embodiments, the cancer is CRC.

According to some embodiments, Fap2 is expressed on the membrane of Fusobacterium. According to certain embodiments, the fusobacterium is fusobacterium nucleatum.

According to some embodiments, the agent that reduces the binding of Fap2 to Gal-GalNAc is selected from the group consisting of: antibody, polypeptide, siRNA, RNAi, and small molecule. Each possibility represents a separate embodiment.

According to some embodiments, the method comprises decreasing the expression of Fap2. According to other embodiments, the method comprises reducing Gal-GalNAc expression on the cancer cells. According to other embodiments, the inhibitor interrupts the binding of Fap2 to Gal-GalNAc.

According to some embodiments, the agent that reduces the binding of Fap2 to Gal-GalNAc inhibits post-translational modification of Fap2.

According to additional embodiments, the agent that reduces the binding of Fap2 to Gal-GalNAc is mutated Fap2.

According to some embodiments, the antibody is against Fap2. According to other embodiments, the antibody is against Gal-GalNAc.

According to some embodiments, the agent that reduces the binding of Fap2 to Gal-GalNAc comprises D-galactose or Gal-GalNAc molecules.

According to some embodiment, the agent that reduces the binding of Fap2 to Gal-GalNAc is administered in conjunction with one or more chemotherapeutic agents, immunotherapeutic agents, surgery or radiotherapy.

According to some embodiments, the agent that reduces the binding of Fap2 to Gal-GalNAc is formulated for sustained release.

According to some embodiments the inhibitor is administered intravenously.

Other objects, features and advantages of the present invention will become clear from the following description and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1H show the expression of Gal-GalNAc in human colorectal adenocarcinoma and specific adenoma subgroups, and facilitates F. nucleatum Enrichment. FIGS. 1A-1B—Gal-GalNAc levels in human colon adenocarcinomas, adenomas, and normal tissues using tissue microarrays (TMA). FIG. 1A—Representative stained TMA images of human colon adenocarcinoma (CRC) and normal tissue (N), H&E (top) and FITC labeled Gal-GalNAc-specific PNA (green) and Hoechst dye (blue, bottom). FIG. 1B—PNA binding to each tissue core (sum of fluorescence intensity of analyzed section; n, number of cases). Error bars indicate mean±SEM. ****p<0.0001, Wilcoxon signed-rank test. FIG. 1C—Gal-GalNAc expression within adenoma subgroups. PNA binding (sum of fluorescence intensity of analyzed section) to the adenoma tissue core presented in FIG. 1B and divided to adenoma groups. Error bars indicate mean±SEM. ****p<0.0001, ANOVA, Tukey's multiple comparison test. FIG. 1D—human colon adenocarcinomas were treated with O-glycanase for Gal-GalNAc removal and stained as described in FIG. 1A. Dashed lines indicate CRC adjacent normal tissue border. FIG. 1E—PNA binding (sum of fluorescence intensity of analyzed field) of samples untreated or treated with O-glycanase. Each symbol represents the mean of three randomly selected fields (n=5 cases). Error bars indicate mean±SEM. *p=0.0313, Wilcoxon signed-rank test. FIG. 1F—Binding of FITC-labeled Fn (single green rods or aggregates seen as green spots) to Hoechst-stained (blue) human colon adenocarcinoma sections. Representative image (left) and magnified inset images (right). FIG. 1G—Quantitation of fusobacterial binding (Fn/mm²) to TMA sections from human colon adenocarcinomas and normal tissues. Symbols represent individual cases. Error bars indicate mean±SEM. ****p<0.0001, one-tailed Mann-Whitney test. FIG. 1H—Quantitation of fusobacterial binding (Fn/mm²) in CRC samples untreated or treated with O-glycanase. Each symbol represents the mean of three randomly selected fields per human section (n=5 cases). Mean±SEM are shown; *p=0.0313, Wilcoxon signed-rank test.

FIG. 2A-2H show that Fap2 binding to GalNAc in human CRC mediates F. nucleatum adenocarcinoma enrichment. FIG. 2A—Fap2 is a Gal-GalNAc binding lectin. Hemagglutination activity is shown by wild-type Fn and not by isogenic Fap2 inactivated mutants K50 and D22 in the absence (left) or in the presence (right) of 25 mM GalNAc. FIG. 2B—Representative image of FITC-labeled Fn (green) attachment to Hoechst-stained (blue) human colon adenocarcinoma sections in the absence (left) or presence (right) 300 mM GalNAc. FIG. 2C—Quantitation of fusobacterial binding (Fn/mm²) performed in FIG. 2B. Each symbol represents the mean of three randomly selected fields per human section (n=6). FIG. 2D—Representative image of Cy3-labeled Fn (red) and Cy5-labeled Fap2-inactivated isogenic mutant K50 (green) to a Hoechst-stained (blue) human colon adenocarcinoma section. FIG. 2E—Quantitation of fusobacterial binding (Fn/mm²) to TMA of human colon adenocarcinoma, adenoma, and normal tissue. Each symbol represents the mean of three randomly selected fields per human tissue core. Mean±SEM are shown; ****p<0.0001, Bonferroni-corrected Wilcoxon test. Mean±SEM are shown; *p=0.015, Wilcoxon signed-rank test. FIG. 2F—Attachment of FITC-labeled (green) Fn (left) or of Fap2-inactivated isogenic mutant D22 (right) to Hoechst-stained (blue) representative human colon adenocarcinoma sections. FIG. 2G—Quantitation of fusobacterial binding (Fn/mm²) described in FIG. 2F. Each symbol represents the mean of three randomly selected fields per human section (n=6). Mean±SEM are shown; *p=0.0119, one-tailed Mann-Whitney test. FIG. 2H—Fn colocalization with Gal-GalNAc in human CRC. Human colorectal adenocarcinoma sections were stained with Hoechst (blue) and incubated with Alexa Fluor 647-conjugated PNA (red) and FITC-labeled Fn (green). Dashed line indicates the CRC-adjacent normal tissue border. Representative image (left). Magnification of the inset CRC region is shown in the middle, and the inset adjacent to normal tissue is shown on the right.

FIGS. 3A-3L show that Fap2-dependent Gal-GalNAc Binding mediates F. nucleatum CRC attachment. Flow cytometry analyses of attachments assays to mouse CRC cell line CT26 and human CRC cell lines HCT116, RKO, and HT29 without and with increasing concentrations of GalNAc. FIGS. 3A-3D—FITC-labeled PNA, Fn, Fap2-inactivated isogenic mutants K50 or D22 in attachment assays to HCT116 (FIG. 3A), CT26 (FIG. 3B), RKO (FIG. 3C) or HT29 (FIG. 3D). FIGS. 3E-3H—FITC-labeled human CRC F. nucleatum isolates CTI-2 and CTI-7 in attachment assays to HCT116 (FIG. 3E), CT26 (FIG. 3F), RKO (FIG. 3G) or HT29 (FIG. 3H). FIGS. 3I-3L—Binding of FITC-labeled F. nucleatum CRC isolates, oral isolates, and an inflammatory bowel disease isolate (as indicated) to mouse CRC cell line CT26 and human CRC cell lines HCT116, RKO, and HT29. Attachment assays were performed to HCT116 (FIG. 3I), CT26 (FIG. 3J), RKO (FIG. 3K) or HT29 (FIG. 3L). For FIGS. 3A-3L—Data reflect three independent experiments. Mean values with SEM of triplicate are shown. Bacterial attachment data in the absence of GalNAc are the mean±SEM of five independent experiments. *p=0.04167, Spearman rank correlation coefficient; **p<0.01, Bonferroni-corrected two-tailed Mann-Whitney test (**p<0.01, ***p=0.0007).

FIGS. 4A-4J show that localization of F. nucleatum to established CRC tumors requires Fap2. FIG. 4A—Experimental scheme: orthotopic rectal CT26 mouse CRC model. When tumors were 2,500 mm³, mice were randomized to a bacterial inoculation group. FIGS. 4B-4C—Gal-GalNAc overexpression in the CT26 mouse CRC model. FIG. 4B—Representative image of CT26 orthotopic tumor stained with H&E or with FITC-labeled Gal-GalNAc-specific PNA (green) and Hoechst dye (blue). CRC denotes images of tumors, and N denotes images of adjacent normal tissue. FIG. 4C—Quantitative analysis of PNA binding to each section (sum of fluorescence intensity of analyzed section). n, number of mice. Error bars indicate mean±SEM. *p=0.0313, Wilcoxon signed-rank test. White arrow indicates tumor, black arrow adjacent normal colon. FIGS. 4D-4E—Preferential enrichments of F. nucleatum ATCC 23726 in CRC tumors. FIG. 4D shows abundance (CFU/gr tissue) and FIG. 4E shows relative fusobacterial gDNA abundance (2^{-ΔCt}) in colon samples from non-CT26 transplanted, tumor-free mice (no CRC), inoculated intravenously (IV) with 5×10⁶ to 1×10⁷ F. nucleatum ATCC 23726, in tumor (T) and normal adjacent tissues (N) from CT26-tumor-bearing mice (n=15) inoculated IV with 5×10⁶ to 1×10⁷ F. nucleatum ATCC 23726 and in tumor (T) and normal adjacent tissues (N) from CT26-tumor-bearing mice (n=15) inoculated IV with 5×10⁶ to 1×10⁷ P. gingivalis ATCC 33277 (Pg). ****p<0.0001,**p<0.01, Mann-Whitney U test; ***p=0.0005, *p<0.05, Bonferroni-corrected Wilcoxon signed-rank test. n.s.—not statistically significant. Each symbol represents data from individual mice. Data reflect one representative experiment out of three performed in FIG. 4B and FIG. 4C and two in FIG. 4D and FIG. 4E. Error bars show mean±SEM. FIGS. 4F-4J—Fap2 mediates fusobacterial localization in CT26 CRC model mice. FIG. 4F—CRC colonization (CFU/gr tissue) by IV inoculated F. nucleatum ATCC 23726 (Fn WT 23726) or Fap2-deficient mutant D22 (MUT D22). ****p<0.0001, Bonferroni-corrected Wilcoxon signed-rank test for (T) versus (N); ****p<0.0001, Mann-Whitney U test for (WT 23726) versus (MUT D22). FIG. 4G Relative fusobacterial gDNA abundance (2^{-ΔCt}) of wild-type Fn (WT) and of the Fap2-deficient isogenic mutant D22 in tumor (T) versus matched adjacent normal tissue (N) from the samples in FIG. 4F. Error bars indicate mean±SEM; ****p<0.0001, ***p=0.0002, Mann-Whitney U test. FIG. 4H—Tumor enrichment of Fn and the Fap2-deficient mutant K50 IV inoculated as a mixture; **p =0.0046, Bonferroni-corrected Wilcoxon signed-rank test. FIGS. 4I and 4J—Tumoral enrichment of inoculated Fap2-expressing CTI-2 or of the Fap2-deficient CTI-7 in tumor (T) and normal tumor-adjacent tissues (N), quantified by plating (FIG. 4I) or by qPCR (FIG. 4J) as relative gDNA abundance in tumor versus matched adjacent normal tissue (2^{-ΔCt}); *p=0.0156, **p=0.0064, Bonferroni-corrected Mann-Whitney U test. Figures show data from one of two representative experiments performed.

FIG. 5A-5E show that fusobacterial presence in CRC metastases is facilitated by Fap2 binding to host Gal-GalNAc. FIG. 5A—Relative fusobacterial (Fn) and P. gingivalis (Pg) gDNA abundance (2^{-ΔCt}) in human CRC metastases and in tumor-free liver biopsy samples. Open circle represents metastasis in the omentum, and open square represents metastasis in the lung. Filled circle are liver metastases. Filled squares represent tumor-free liver. Error bars indicate mean±SEM; **p=0.004, Bonferroni corrected Wilcoxon signed-rank test; *p=0.031, Bonferroni-corrected Mann-Whitney U test. Each symbol represents data from individual metastatic deposits. FIG. 5B—Representative sections of human CRC metastases (M) were stained with FITC-PNA (green) for Gal-GalNAc quantification and with Hoechst (blue). Dashed lines indicate tumor-adjacent normal (N) tissue border. FIG. 5C—Quantitative analysis of PNA binding (sum of fluorescence intensity of analyzed field) of the samples described in FIG. 5B. Each symbol represents the mean of three randomly selected fields for each human tissue section (n=9). Error bars indicate mean±SEM; **p=0.0039, Wilcoxon signed-rank test. FIG. 5D—Attachment of Cy3-labeled (red) Fn (F. nucleatum) (Fn and of its Cy5-labeled (green) Fap2-inactivated mutant K50 to a representative Hoechst-stained (blue) human CRC liver metastasis section. FIG. 5E—Quantitation of fusobacterial binding (Fn/mm²) of bacteria described in FIG. 5D to sections of human CRC metastasis sections (n=8). Each symbol represents the median of three randomly selected fields per human section. Error bars indicate mean±SEM; **p=0.0078, Wilcoxon signed-rank test.

FIG. 6 summarizes the hemagglutination activity (indicating Fap2 presence), presence of Fap2 in the strain's genome, sub-species designation, and source and/or reference for the fusobacterial strains that were used.

FIGS. 7A-7B show images of representative tumors displaying high and low Gal-GalNAc levels. Tissue microarray (TMA) (Boimax inc.: MC5003b, MC2082a, BN1002b) were used to quantify Gal-GalNAc in tumor and matching normal control sections. Lung (top) and pancreas (Bottom) adenocarcinomas displaying high Gal-GalNAc levels are presented in FIG. 7A. Sarcoma (top) and hepatocellular liver cancer (bottom) non-adenocarcinoma tumors displaying low Gal-GalNAc levels are shown in FIG. 7B. Left panels present H&E staining. Middle and right panels present FITC-labeled Gal-GalNAc-specific PNA (green) and Hoechst dye (blue) of tumor (middle panel) and normal (right panel). Bars shown are 250 μm scale.

FIGS. 8A-8B show that High Gal-GalNAc levels are displayed in human adenocarcinomas. FIG. 8A—Tumors were arranged according to increasing Gal-GalNAc levels. FIG. 8B—Gal-GalNAc levels of the tumors described in FIG. 8A were compared to matching normal tissue controls (open symbols). The normal tissue controls for esophagus, lung and skin were used twice for the respective esophagus adenocarcinoma and squamous cell carcinoma (SCC); the respective lung adenocarcinoma and SCC, and for the melanoma and SCC. Each symbol represents the fluorescent intensity of a different sample. Error bars indicate mean±SEM. *p<0.05, **p<0.01, ***p=0.0001 Two-tailed Mann-Whitney test. Adenocarcinoma vs. non-adenocarcinoma was calculated using two-tailed t-test p<0.0001.

### DETAILED DESCRIPTION OF THE INVENTION

The present invention provides methods and compositions for treating and diagnosing cancer. The invention is based on the unexpected discovery that Fap2 mediates the attachment of Fusobacteria to cancer cells. Fap2 interacts with the Gal-GalNAc molecules that are displayed on the cancer cells. It is now disclosed that high Gal-GalNAc levels exist in a variety of adenocarcinomas such as, stomach, prostate, ovary, colon, uterus, pancreas, breast, lung and esophagus, which offer a valuable target for therapeutics. The present invention provides inhibition of Fap2/Gal-GalNAc interaction for cancer therapy. It is now further disclosed that Fap2 may be employed as a targeting moiety to cancer cells. For example, Fap2 may target fusobacteria that were engineered to serve as a platform for treating CRC and other adenocarcinomas.

The binding of fusobacterium to CRC is mediated by Fap2 lectin that binds to D-galactose-β(1-3)-N-acetyl-D-galactosamine (Gal-GalNAc). High Gal-GalNAc levels were also detected in CRC metastases and correlated with fusobacterial gDNA occurrence in these metastases (Abed et al., 2016; Cell Host & Microbe 20, 215-225), demonstrating the capacity of fusobacteria to colonize CRC metastases.

The term “Fap 2” as used herein refers to the outer membrane protein of Fusobacterium nucleatum. The fap2 gene encodes 3,692 amino acids, resulting in a very large OMP with a predicated molecular mass of 390 kDa. The present invention comprises Fap2 homologues that possesses the activity of binding Gal-GalNAc. An exemplary Fap2 according to the invention is set forth in GenBank accession number: EDK89413.

According to an aspect, the present invention provides a method of modulating the interaction between Gal-GalNAc present on cancer cells and a Fap2 protein, said modulating is selected from the group consisting of:
(i) inhibiting or reducing the interaction between Fap2 and Gal-GalNAc; and
(ii) utilizing Fap2 as a targeting moiety to Gal-GalNAc presented on cancer cells.

According to an aspect, the present invention provides a method of targeting a therapeutic, imaging, or diagnostic agent to a tumor in a subject, the method comprising administering to the subject a composition comprising:
(i) a Fap2 protein or a fragment thereof; and
(ii) the therapeutic, imaging, or diagnostic agent, wherein said cancer is characterized by elevated amounts of Gal-GalNAc.

The term “elevated amounts of Gal-GalNAc” as used herein refers to higher amounts of Gal-GalNAc molecules, and/or to higher amounts of Gal-GalNAc molecules that are being exposed at the external cancer cell surface. The terms “overexpress Gal-GalNAc”, “over display Gal-GalNAc”, or “over present Gal-GalNAc” are used herein interchangeably and refer to higher number of Gal-GalNAc molecules and/or to higher number of exposed Gal-GalNAc molecules on cancer cells compared to the corresponding normal cells.

The term “fragment thereof comprising a Gal-GalNAc binding site” refers to a fragment of Fap2 that is capable of binding Gal-GalNAc molecules. According to some embodiments, Gal-GalNAc amount is elevated in cancer cells by at least 10%, 20%, 30%, 40%, 50%, 60%, 70%, 80%, 90%, 100%, 200%, 300%, 400% or 500% compared to a normal or control cell. Each possibility represents a separate embodiment of the invention. According to additional embodiments, Gal-GalNAc amount is elevated in cancer cells by at least 2-, 3-, 4-, 5-, 6-, 7-, 8-, 9-, or 10-fold compared to normal or control cells. Each possibility represents a separate embodiment of the invention.

According to some embodiments, the therapeutic agent is an immunotherapeutic agent. According to certain embodiments, the therapeutic agent is a chemotherapeutic agent.

According to some embodiments, the therapeutic agent is a polypeptide capable of inducing cell death in the cancer cell. According to other embodiments, the therapeutic agent is a toxin.

According to some embodiments, the therapeutic agent is a modified or genetic engineered Fusobacterium. According to certain embodiments, the modified Fusobacterium comprises a toxin. According to additional embodiments, the Fap2 is expressed by the Fusobacterium. According to certain embodiments, the Fap2 is engineered not to bind or activate TIGIT. According to certain embodiments, the Fusobacterium is Fusobacterium nucleatum.

TIGIT expression on NK cells serves, inter alia, as the receptor that binds the Fap2 protein of Fusobacterium nucleatum. The interaction between F. Nucleatum and TIGIT leads to reduced NK cytotoxic activity (Gur et al., Immunity 42, 344-355, 2015). It will be, therefore, advantageous to engineer Fap2 not to bind or activate TIGIT.

According to some embodiments, the therapeutic agent is within a liposome. According to certain embodiments, the liposome presents at least one Fap2 molecule on its membrane. According to certain embodiments, the therapeutic agent is embedded within microcapsules, liposomes, microemulsions, or microspheres.

According to some embodiments, Fap2 is directly coupled to the therapeutic agent, diagnostic, or imaging agent. According to some embodiments, Fap2 is coupled to the therapeutic, diagnostic or imaging agent through a linker. According to other embodiments, Fap2 is associated with the therapeutic, diagnostic or imaging agent.

According to some embodiments, the composition further comprises an antibody against Fap2 protein.

According to some embodiments the Fap2 fragment comprises Gal-GalNAc binding site. According to additional embodiments, the Fap2 fragment comprises the pharmacophore that enable binding of Fap2 to Gal-GalNAc.

The pharmacophore is an ensemble of steric and electronic features that is necessary to ensure the optimal supramolecular interactions with a specific biological target and to trigger (or block) its biological response. A pharmacophore that retains the activity of Fap2 or fragment thereof as described herein is also include in the present invention.

According to some embodiments, the therapeutic agent, diagnostic or imaging agent comprises a radioisotope.

According to some embodiments, the imaging agent is selected from the group consisting of: fluorescent, radio-imaging agent, and an agent used to enhance CT, MRI, or ultrasound imaging.

According to some embodiment, the method further comprising treating with an additional anticancer therapy. According some embodiments, the anticancer therapy is selected from surgery, radiotherapy and/or chemotherapy. According to certain embodiments, the anticancer therapy is an anti-cancer agent.

According to some embodiments, the composition is a pharmaceutical composition further comprising a pharmaceutically acceptable carrier.

According to some embodiments, the subject is human. According to additional embodiments, the subject is animal.

According to an additional aspect, the present invention provides a method of treating cancer, the method comprising administering to a subject in need thereof a therapeutically effective amount of at least one inhibitor agent that reduces the binding of Fap2 to Gal-GalNAc.

According to some embodiments, Fap2 is expressed on the membrane of Fusobacterium. According to certain embodiments, the fusobacterium is fusobacterium nucleatum. According to some embodiments, the Gal-GalNAc is displayed on the membrane of the cancer cells.

The terms “inhibitor” and “agent that reduces the binding” are used herein interchangeably and refer to an agent or compound capable of inhibiting the interaction or complexing of Fap2 and Gal-GalNAc. The term “inhibit” is used interchangeably with “reduce” and “block”, and does not require absolute inhibition. According to some embodiments, the inhibitor is selected from the group consisting of: a chemical agent or moiety, a protein, a polypeptide or a peptide, and a polynucleotide molecule. Each possibility represents a separate embodiment of the invention. The scope of the present invention encompasses homologs, analogs, variants and derivative of said inhibitor, with the stipulation that these variants and/or modifications must inhibit or reduce Fap2 interaction with Gal-GalNAc.

The term “binding” refers to the adherence of molecules to one another. The term “subject” includes humans and animals afflicted with cancer and human or animals amenable to therapy with the pharmaceutical compositions described herein. According to additional embodiments, the subject is a subject suspected of having cancer.

As use herein, the terms “administration of” and/or “administering” a composition should be understood to mean providing a compound of the invention or a prodrug of a compound of the invention to a subject in need of treatment. The terms also refer to providing a compound of the invention, e.g. comprising an imaging or diagnostic agent, to a subject suspected of having cancer.

According to some embodiments, the inhibitor is selected from the group consisting of: antibody, polypeptide, siRNA, RNAi, and small molecule. Each possibility represents a separate embodiment.

“RNA interference (RNAi)” is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene, thereby inhibiting expression of the target gene. In some embodiments, the RNA is double stranded RNA (dsRNA). This process has been described in plants, invertebrates, and mammalian cells. In nature, RNAi is initiated by the dsRNA-specific endonuclease Dicer, which promotes cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target genes. As used herein, inhibition by RNAi includes any decrease in expression or protein activity or level of the FAP2 gene or protein encoded by the target gene, i.e., a Fap2. The decrease may be of at least 30%, 40%, 50%, 60%, 70%, 80%, 90%, 95% or 99% or more as compared to the expression of a target gene or the activity or level of the protein encoded by a target gene which has not been targeted by an RNA interfering agent.

“Short interfering RNA” (siRNA), also referred to herein as “small interfering RNA” is defined as an agent which functions to inhibit expression of a target gene, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA). According to other embodiment, an siRNA is a small hairpin (also called stem loop) RNA (shRNA). In some embodiments, these shRNAs are composed of a short (e.g., 19-25 nucleotide) antisense strand, followed by a 5-9 nucleotide loop, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501). RNA interfering agents, e.g., siRNA molecules, may be administered to a subject having or at risk for having cancer, to inhibit expression of FAP2, and thereby treat, ameliorate, or inhibit cancer in the subject.

According to some embodiments, administering the pharmaceutical composition of the invention to a subject may increase the progression-free survival of the treated subject by 10% or more, e.g., 15% or more, 20% or more, 25% or more, 30% or more, 40% or more, or 50% or more, and may increase the progression-free survival of patients diagnosed with the tumor by 100% or less, e.g., 90% or less, 80% or less, 70% or less, 60% or less, or 50% or less, compared to a non-treated subject. In certain embodiments, administering the pharmaceutical composition of the invention to the subject in need may increase the progression-free survival of the subject by a range of 10 to 100%, e.g., 15 to 95%, 20 to 90%, 25 to 85%, 30 to 80%, including 40 to 70% compared to a non-treated subject.

According to some embodiments, the treatment comprises decreasing the expression of Fap2. According to other embodiments, the treatment comprises reducing Gal-GalNAc expression on the cancer cells. According to additional embodiments, the inhibitor interrupts the binding of Fap2 to Gal-GalNAc.

According to some embodiments, the antibody is against Fap2. According to other embodiments, the antibody is against Gal-GalNAc.

The term “antibody” is used in the broadest sense and includes monoclonal antibodies (including full length or intact monoclonal antibodies), polyclonal antibodies, multivalent antibodies, and antibody fragments long enough to exhibit the desired biological activity. According to some embodiments, the antibody inhibits the binding of Fap2 to Gal-GalNAc. According to additional embodiments of the invention, the antibody is against Fap2-Gal-GalNAc binding domain.

Antibody or antibodies according to the invention include intact antibodies, such as polyclonal antibodies or monoclonal antibodies (mAbs), as well as proteolytic fragments thereof, such as the Fab or F(ab′)2 fragments. Single chain antibodies also fall within the scope of the present invention.

“Antibody fragments” comprise only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment (Ward et al., Nature 1989, 341, 544-546) which consists of a VH domain; (vii) isolated CDR regions; (viii) F(ab′)₂ fragments, a bivalent fragment including two Fab′ fragments linked by a disulphide bridge at the hinge region; (ix) single chain antibody molecules (e.g. single chain Fv; scFv) (Bird et al., Science 1988, 242, 423-426; and Huston et al., Proc. Natl. Acad. Sci. (USA) 1988, 85, 5879-5883); (x) “diabodies” with two antigen binding sites, comprising a heavy chain variable domain (VH) connected to a light chain variable domain (VL) in the same polypeptide chain (see, e.g., EP 404,097; WO 93/11161; and Hollinger et al., Proc. Natl. Acad. Sci. USA, 1993, 90, 6444-6448); (xi) “linear antibodies” comprising a pair of tandem Fd segments (VH-CH1-VH-CH1) which, together with complementary light chain polypeptides, form a pair of antigen binding regions (Zapata et al. Protein Eng., 1995, 8, 1057-1062; and U.S. Pat. No. 5,641,870).

According to some embodiments, the inhibitor comprises D-galactose or Gal-GalNAc molecules. According to some embodiments, the inhibitor is a Fap2 peptide from the binding domain of the Fap2 protein that binds to Gal-GalNAc.

According to some embodiments, the agent is formulated for sustained release.

According to some embodiment, the inhibitor is administered in conjunction with one or more chemotherapeutic agents, immunotherapeutic agents, or radiotherapy.

According to an additional aspect, the present invention provides a composition comprising:
(i) a Fap2 protein or a fragment thereof; and
(ii) the therapeutic, diagnostic or imaging agent.

The Fap2, Fap2 fragment, and the therapeutic, diagnostic or imaging agent are as described hereinabove.

According to an aspect, the present invention provides a method of diagnosing cancer in a subject, said cancer is characterized by elevated amounts of Gal-GalNAc molecules on cell surface, the method comprising determining the expression level of Gal-GalNAc in a biological sample of said subject using a composition according to the invention and comparing the expression level of Gal-GalNAc with a control or a reference sample.

According to some embodiments, the Fap2 is a mutated protein. According to certain embodiment, mutated Fap2 have at least 85%, 86%, 87%, 88%, 89%, 90%, 91%, 92%, 93%, 94%, 95%, 96%, 97%, 98%, or 99% homology to Fap2. Each possibility represents a separate embodiment of the present invention.

### Pharmacology

In pharmaceutical and medicament formulations, the active agent is preferably utilized together with one or more pharmaceutically acceptable carrier(s) and optionally any other therapeutic ingredients. The carrier(s) must be pharmaceutically acceptable in the sense of being compatible with the other ingredients of the formulation and not unduly deleterious to the recipient thereof. The active agent is provided in an amount effective to achieve the desired pharmacological effect, as described above, and in a quantity appropriate to achieve the desired exposure.

The pharmaceutical compositions of the invention may be formulated to control release of active ingredient or to prolong its presence in a patient's system. Numerous suitable drug delivery systems are known and include, e.g., implantable drug release systems, hydrogels, hydroxymethylcellulose, microcapsules, liposomes, microemulsions, microspheres, and the like. Controlled release preparations can be prepared through the use of polymers to complex or adsorb the molecule according to the present invention. For example, biocompatible polymers include matrices of poly(ethylene-co-vinyl acetate) and matrices of a polyanhydride copolymer of a stearic acid dimer and sebacic acid. The rate of release of the molecules according to the present invention from such a matrix depends upon the molecular weight of the molecule, the amount of the molecule within the matrix, and the size of dispersed particles.

The composition of this invention may be administered by any suitable means, such as orally, topically, intranasally, subcutaneously, intramuscularly, intravenously, intra-arterially, intraarticulary, intralesionally, intratumorally or parenterally. According to certain embodiments, the composition is administered intravenously.

It will be apparent to those of ordinary skill in the art that the therapeutically effective amount of the pharmaceutical compositions according to the present invention will depend, inter alia upon the administration schedule, the unit dose of composition administered, whether the composition is administered in combination with other therapeutic agents, the immune status and health of the patient, the therapeutic activity of the composition administered, its persistence in the blood circulation, and the judgment of the treating physician.

The term “therapeutic agent,” as used herein, is defined as any substance intended for use in the treatment of cancer in an animal, preferably in a human. The term therapeutic agent includes active, activated and metabolized forms of therapeutic agents. The term “therapeutic agent” includes a substance that is being activated after administration with, for example, heat, light, or radiation (e.g., quantum dot).

As used herein the term “therapeutically effective amount” refers to an amount of a drug effective to treat a disease or disorder in a mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

The cancer amendable for treatment by the present invention includes any cancer that is characterized by high levels of Gal-GalNAc. According to some embodiments, the cancer is carcinoma. According to some embodiment, the cancer is adenocarcinoma. According to some embodiments, the adenocarcinoma is selected from the group consisting of: breast, esophagus, uterus, pancreas, prostate, lung, colon, stomach, ovary, and cervix adenocarcinomas.

According to certain embodiments, the cancer is selected from the group consisting of adrenocortical carcinoma (ACC), colon and rectal adenocarcinoma (COAD, READ), pancreatic ductal adenocarcinoma (PAAD), lung adenocarcinoma (LUAD), prostate adenocarcinoma

(PRAD), ovarian serous cystadenocarcinoma (OV). Each possibility represents a separate embodiment of the invention.

The molecules of the present invention as active ingredients are dissolved, dispersed or admixed in an excipient that is pharmaceutically acceptable and compatible with the active ingredient as is well known. Suitable excipients are, for example, water, saline, phosphate buffered saline (PBS), dextrose, glycerol, ethanol, or the like and combinations thereof. Other suitable carriers are well known to those skilled in the art. In addition, if desired, the composition can contain minor amounts of auxiliary substances such as wetting or emulsifying agents, and/or pH buffering agents.

The term “treatment” as used herein refers to both therapeutic treatment and prophylactic or preventative measures.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia.

According to some embodiments, the method of treating cancer comprises administering a pharmaceutical composition as part of a treatment regimen comprising administration of at least one additional anti-cancer agent.

According to some embodiments, the anti-cancer agent is selected from the group consisting of an antimetabolite, a mitotic inhibitor, a taxane, a topoisomerase inhibitor, a topoisomerase II inhibitor, an asparaginase, an alkylating agent, an antitumor antibiotic, and combinations thereof. Each possibility represents a separate embodiment of the invention.

According to some embodiments, the antimetabolite is selected from the group consisting of cytarabine, gludarabine, fluorouracil, mercaptopurine, methotrexate, thioguanine, gemcitabine, and hydroxyurea. According to some embodiments, the mitotic inhibitor is selected from the group consisting of vincristine, vinblastine, and vinorelbine. According to some embodiments, the topoisomerase inhibitor is selected from the group consisting of topotecan and irenotecan. According to some embodiments, the alkylating agent is selected from the group consisting of busulfan, carmustine, lomustine, chlorambucil, cyclophosphamide, cisplatin, carboplatin, ifosamide, mechlorethamine, melphalan, thiotepa, dacarbazine, and procarbazine. According to some embodiments, the antitumor antibiotic is selected from the group consisting of bleomycin, dactinomycin, daunorubicin, doxorubicin, idarubicin, mitomycin, mitoxantrone, and plicamycin. According to some embodiments, the topoisomerase II is selected from the group consisting of etoposide and teniposide. Each possibility represents a separate embodiment of the present invention.

According to some particular embodiments, the additional anti-cancer agent is selected from the group consisting of bevacizumab, carboplatin, cyclophosphamide, doxorubicin hydrochloride, gemcitabine hydrochloride, topotecan hydrochloride, thiotepa, and combinations thereof. Each possibility represents a separate embodiment of the present invention.

According to some embodiments, the anti-cancer agent is an immuno-modulator, whether agonist or antagonist, such as antibody against an immune checkpoint molecule.

According to other embodiments the additional anti-cancer agent is a chemotherapeutic agent. The chemotherapy agent, which could be administered together with the composition according to the present invention, or separately, may comprise any such agent known in the art exhibiting anticancer activity, including but not limited to: mitoxantrone, topoisomerase inhibitors, spindle poison vincas: vinblastine, vincristine, vinorelbine (taxol), paclitaxel, docetaxel; alkylating agents: mechlorethamine, chlorambucil, cyclophosphamide, melphalan, ifosfamide; methotrexate; 6-mercaptopurine; 5-fluorouracil, cytarabine, gemcitabin; podophyllotoxins: etoposide, irinotecan, topotecan, dacarbazin; antibiotics: doxorubicin (adriamycin), bleomycin, mitomycin; nitrosoureas: carmustine (BCNU), lomustine, epirubicin, idarubicin, daunorubicin; inorganic ions: cisplatin, carboplatin; interferon, asparaginase; hormones: tamoxifen, leuprolide, flutamide, and megestrol acetate.

According to some embodiments, the chemotherapeutic agent is selected from the group consisting of alkylating agents, antimetabolites, folic acid analogs, pyrimidine analogs, purine analogs and related inhibitors, vinca alkaloids, epipodophyllotoxins, antibiotics, L-asparaginase, topoisomerase inhibitor, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. According to another embodiment, the chemotherapeutic agent is selected from the group consisting of 5-fluorouracil (5-FU), leucovorin (LV), irenotecan, oxaliplatin, capecitabine, paclitaxel and doxetaxel. One or more chemotherapeutic agents can be used.

Toxicity and therapeutic efficacy of the compositions described herein can be determined by standard pharmaceutical procedures in cell cultures or experimental animals, e.g., by determining the IC50 (the concentration which provides 50% inhibition) and the maximal tolerated dose for a subject compound. The data obtained from these cell culture assays and animal studies can be used in formulating a range of dosages for use in humans. The dosage may vary depending inter alia upon the dosage form employed, the dosing regimen chosen, the composition of the agents used for the treatment and the route of administration utilized, among other relevant factors. The exact formulation, route of administration and dosage can be chosen by the individual physician in view of the patient's condition. Depending on the severity and responsiveness of the condition to be treated, dosing can also be a single administration of a slow release composition, with course of treatment lasting from several days to several weeks or until cure is effected or diminution of the disease state is achieved. The amount of a composition to be administered will, of course, be dependent on the subject being treated, the severity of the affliction, the manner of administration, the judgment of the prescribing physician, and all other relevant factors.

The term “administering” or “administration of” a substance, a compound or an agent to a subject can be carried out using one of a variety of methods known to those skilled in the art. For example, a compound or an agent can be administered enterally or parenterally. Enterally refers to administration via the gastrointestinal tract including per os, sublingually or rectally. Parenteral administration includes administration intravenously, intradermally, intramuscularly, intraperitoneally, subcutaneously, ocularly, sublingually, intranasally, by inhalation, intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. In some embodiments, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

The term “about” means that an acceptable error range, e.g., up to 5% or 10%, for the particular value should be assumed.

### Diagnosis

The present invention further discloses methods for diagnosing and prognosing cancer.

According to an aspect, the present invention provides a diagnostic and/or prognostic method of cancer in a subject, the method comprises the step of determining the level of Gal-GalNAc in a biological sample of said subject using at least one Fap2 or part thereof as described herein.

According to an additional aspect, the present invention provides a method of treating a cancer characterized by elevated amounts of Gal-GalNAc in a subject, the method comprising the steps of: (i) administering to the subject the composition comprising the diagnostic agent as described herein; and (ii) treating said subject with an anti-cancer therapy.

According to an additional aspect, the present invention provides a method of treating a cancer characterized by elevated amounts of Gal-GalNAc in a subject, the method comprising the steps of: (i) determining the expression level of Gal-GalNAc in a biological sample of said subject using the composition as described herein; and (ii) treating said subject with an anti-cancer therapy.

According to some embodiments, the method comprising administering to the subject a therapeutically effective amount of the pharmaceutical composition as described hereinabove.

The term “biological sample” encompasses a variety of sample types obtained from an organism that may be used in a diagnostic or monitoring assay. The term encompasses blood and other liquid samples of biological origin, solid tissue samples, such as a biopsy specimen, or tissue cultures or cells derived there from and the progeny thereof. Additionally, the term may encompass circulating tumor or other cells. The term specifically encompasses a clinical sample, and further includes cells in cell culture, cell supernatants, cell lysates, serum, plasma, urine, amniotic fluid, biological fluids including aqueous humour and vitreous for eyes samples, and tissue samples. The term also encompasses samples that have been manipulated in any way after procurement, such as treatment with reagents, solubilization, or enrichment for certain components.

Also provided herein are kits that find use in practicing the subject methods, as described herein. In certain embodiments, a subject kit may include an agent, e.g., an antibody, polypeptide, small molecule, nucleic acid, etc., as described above, that inhibits the binding of Fap2 to Gal-GalNAc molecules for administering to a subject with a cancer characterized by elevated amounts of Gal-GalNAc. In certain embodiments, a subject kit may be provided with other active agents, e.g., anti-cancer drugs, to be co-administered with the agent that inhibits said binding.

In certain embodiments, a subject kit includes instructions for carrying out the subject methods, as discussed above, which are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or sub-packaging) etc. In other embodiments, the instructions are present as an electronic storage data file present on a suitable computer-readable storage medium, e.g., a digital storage medium, e.g., a CD-ROM, USB drive, Flash drive, etc. The instructions may take any form, including complete instructions for how to use the element(s) of the kit, or as a website address with which instructions posted on the Internet may be accessed.

The following examples are presented in order to more fully illustrate some embodiments of the invention. They should, in no way be construed as limiting the scope of the invention.

### EXAMPLES

### Experimental Procedures

Collection of Clinical Samples—The Hadassah Medical School institutional review board approved the use of human samples for this study. Informed consent was obtained from all patients. CRC metastases from five frozen and seven formalin-fixed paraffin embedded blocks were collected from the Israel Collaborative Biorepository for Research (MIDGAM). Seven tumor-free liver tissue samples were collected from the pathology department at Hadassah Medical School.

Tissue Microarray Analysis—Colon cancer tissue array CO2601 (US Biomax) and array CO809a (US Biomax) were used in these studies. Details about the cases for each core on the array are available on the US Biomax Web site.

Bacterial Strains and Growth Conditions—F. nucleatum strains ATCC 23726, K50, D22, ATCC 10953, PK 1594, CTI-1, CTI-2, CTI-3, CTI-5, CTI-6, CTI-7, EAVG 002, and P. gingivalis ATCC 33277 were cultured as described in Abed et al. 2016. ibid, Supplemental Experimental Procedures. Regarding the use of the K50 and D22, two mutants strains derived from ATCC 23726 with a disrupted and inactive fap2 gene (Coppenhagen-Glazer et al., 2015, ibid), given the similarity in phenotype these strains are used interchangeably in subsequent experiments.

Cell Lines and Tissue Culture—CT26 stably transfected with the luciferase (luc) gene (CT26-luc), the human colon adenocarcinoma cell line HT29, RKO, and HCT116 were cultured according to ATCC guidelines.

Murine CRC Model—All experiments were performed in accordance with the guidelines of our institution's animal welfare committee. The orthotopic rectal cancer model was performed as described (Kolodkin-Gal et al., 2009, ibid) in wild-type BALB/cJ mice. Mice were injected with 1×10⁶ CT26-luc cells. Tumor size assessment was performed ad described in Abed et al. 2016. ibid, Supplemental Experimental Procedures.

Bacterial Inoculations—Mice were inoculated with 5×10⁶ to 1×10⁷ bacteria (washed with PBS twice) via tail vein injection. For C57BL/6J wild-type and Apc^{min+/-} mice, mice were aged beyond 12 weeks and then intravenously injected with ˜5×10⁸ prewashed bacteria.

Quantification of Bacteria Using Plating and qPCR—Tissue samples were homogenized using a Fastprep (MP Biomedicals) and plated as described in Abed et al. 2016. ibid, Supplemental Experimental Procedures. Colonies were enumerated after 6 days of incubation under anaerobic conditions. DNA preparation and qPCR of homogenized tissue are as described in Abed et al. 2016. ibid, Supplemental Experimental Procedures.

Flow Cytometry and Competition Assays—FITC-labeled F. nucleatum were incubated with cells at a MOI of 10 for 30 min at room temperature. FITC-labeled PNA lectin (Sigma-Aldrich) was incubated at a final concentration of 140 nM per 2.5×10⁵ cells. For competition experiments, bacteria or PNA was incubated with GalNAc (concentration range 0, 50, 100, and 300 mM) for 30 min prior to incubation with cells. Flow cytometry methods and analysis are as described in Abed et al. 2016. ibid, Supplemental Experimental Procedures.

Immunofluorescence and Section Preparation—Fixed tissue sections were stained with H&E or processed for immunofluorescence microscopy. Sections were blocked and incubated with fluorescent PNA or fluorescent bacteria and imaging analysis are as described in Abed et al. 2016. ibid, Supplemental Experimental Procedures. GalNAc removal was performed by incubating sections with O-glycanase.; see Supplemental Experimental Procedures for experimental details.

Hemagglutination Assays—Hemagglutination assays were performed as previously described (Coppenhagen-Glazer et al., 2015, ibid). For inhibition assays, washed bacteria were preincubated with 25 mM GalNAc (Sigma-Aldrich) for 30 min prior to incubation with erythrocytes.

Statistical Analysis—GraphPad Prism software version 6.0 was used for statistical analysis. Statistical tests used are indicated in the figure legends.

### Example 1

### F. nucleatum Binds to Gal-GalNAc Overexpressed on CRC

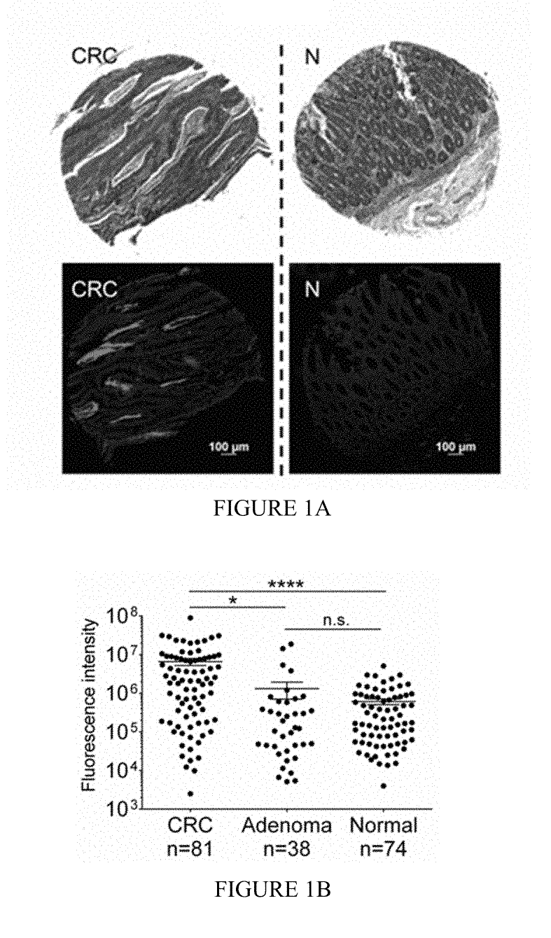

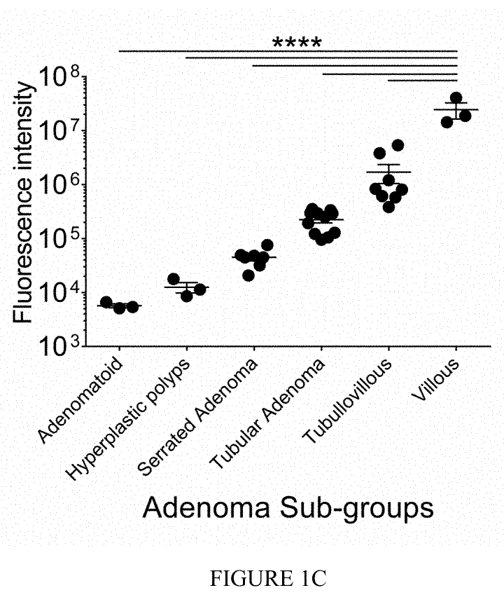

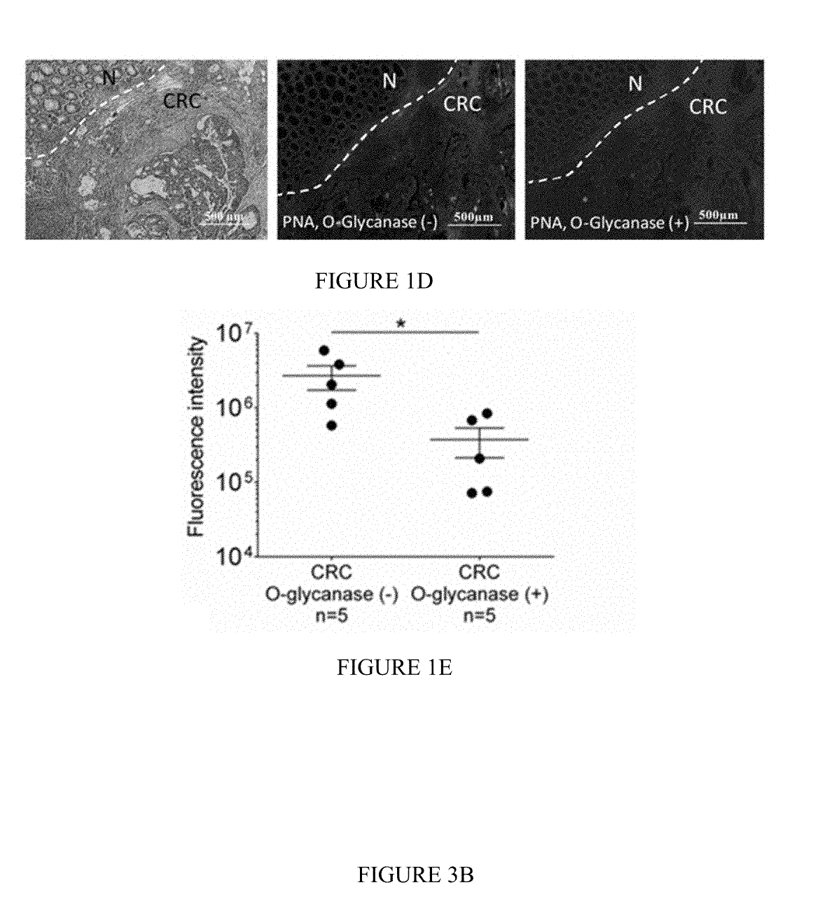

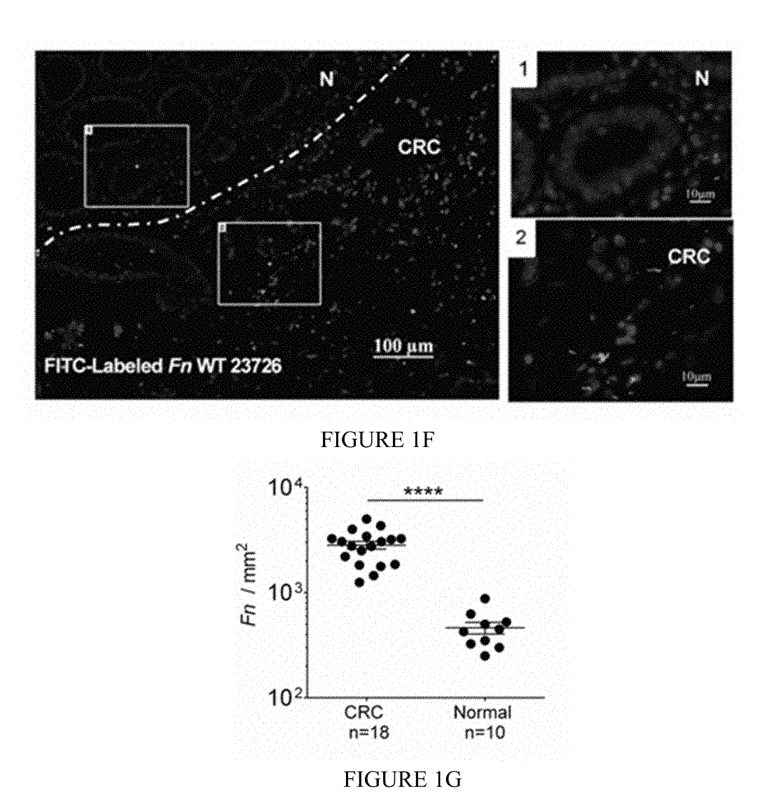

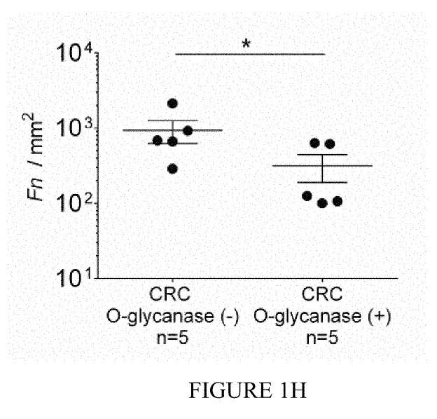

Gal-GalNAc was shown before to be expressed at high levels by adenocarcinomas. These observations led to the hypothesis that colorectal adenocarcinoma expression of Gal-GalNAc may facilitate binding of fusobacteria to CRC. To test this hypothesis, Gal-GalNAc levels on healthy human colorectal tissues, human colonic adenomas, and human colorectal adenocarcinomas was assessed by staining tissue microarrays with FITC-labeled peanut agglutinin (PNA), a Gal-GalNAc [Gal−β(1→3)GalNAc] specific lectin. Gal-GalNAc levels were significantly higher in adenocarcinomas compared to adenomas (FIGS. 1A-1B). Intense staining was detected in the adenocarcinoma's epithelial cells, with some variation of staining intensity across tumoral epithelial cell due to plane of section (FIG. 1A). While adenomas overall seemed to express levels of Gal-GalNAc similar to healthy tissues (FIG. 1B), when the histopathology of the adenomas is considered in more detail, statistically significant trends emerged within the adenoma group. Within the experimental dataset, the highest levels of Gal-GalNAc expression were found on villous adenomas followed by tubulous villous adenomas (14-fold difference, p<0.0001 ANOVA, Tukey's multiple comparison test). Gal-GalNAc differed by 100-fold between villous and tubular adenomas (p<0.0001 ANOVA, Tukey's Multiple Comparison test). Levels of Gal-GalNAc staining were markedly lower on adenomatoid, hyperplastic, and serrated adenomas (FIG. 1C). Notably, of these histopathologic subtypes, the villous growth pattern of adenomas has the highest malignant potential.

To determine if colorectal adenocarcinoma Gal-GalNAc levels may affect F. nucleatum enrichment, O-glycanase ability to reduce Gal-GalNAc levels in human colorectal adenocarcinoma tissue sections was tested. O-glycanase treatment of the human CRC adenocarcinoma sections reduced FITC-PNA staining by nearly 7-fold (FIGS. 1D-1E). Next, a method to visualize binding of F. nucleatum ATCC 23726 (Fn) to formalin-fixed paraffin-embedded human adenocarcinoma samples (FIG. 1F) was developed. Fn binding to adenocarcinoma versus normal colonic tissues correlated with Gal-GalNAc expression levels and increased 6.1 fold in the colonic adenocarcinoma tissues relative to normal tissue (p<0.0001,FIG. 1 F-G). Similar to the observations with O-glycanase treatment and FITC-PNA binding, fusobacterial attachment to the colorectal adenocarcinoma specimens decreased in O-glycanase-treated sections (2.96 fold less; p=0.0313, FIG. 1H). These results suggest that F. nucleatum enrichment depends on Gal-GalNAc levels.

### Example 2

### Fap2 Mediates Attachment of F. nucleatum to Gal-GalNAc Overexpressed in CRC

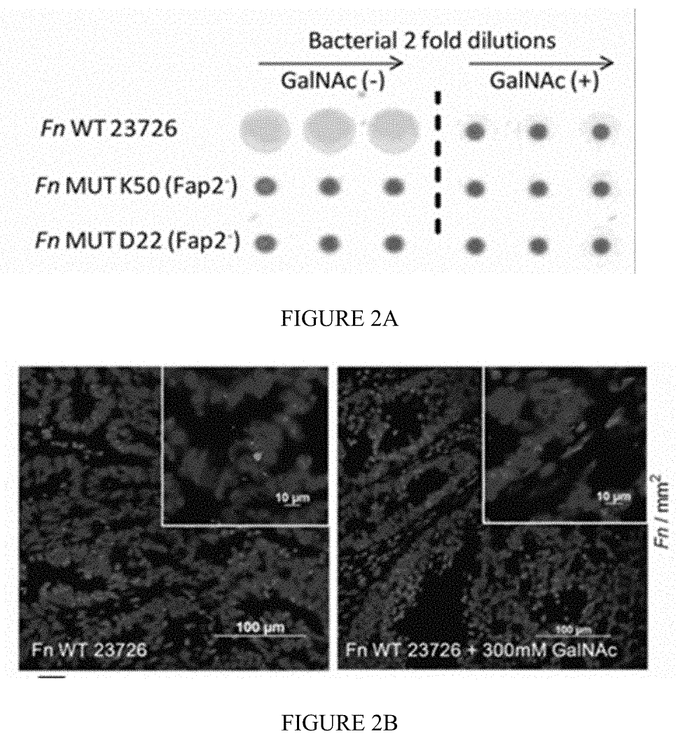

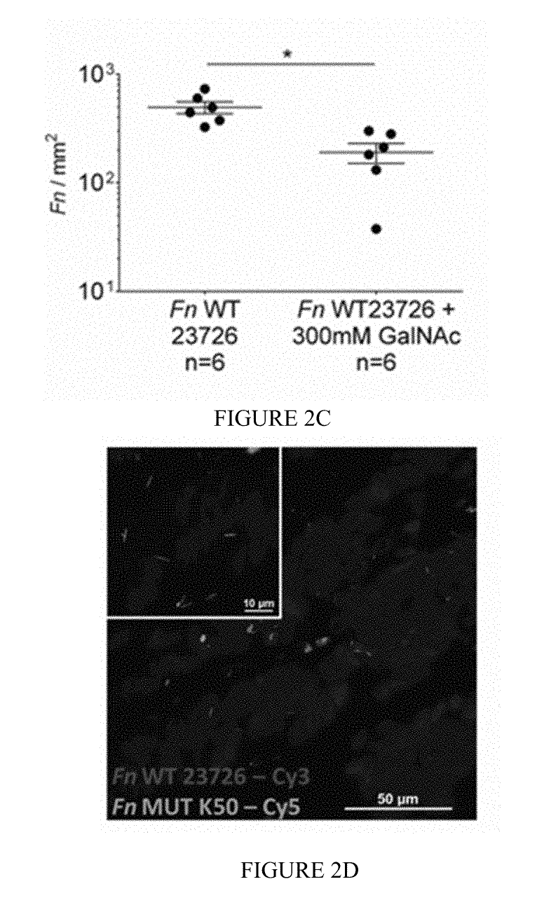

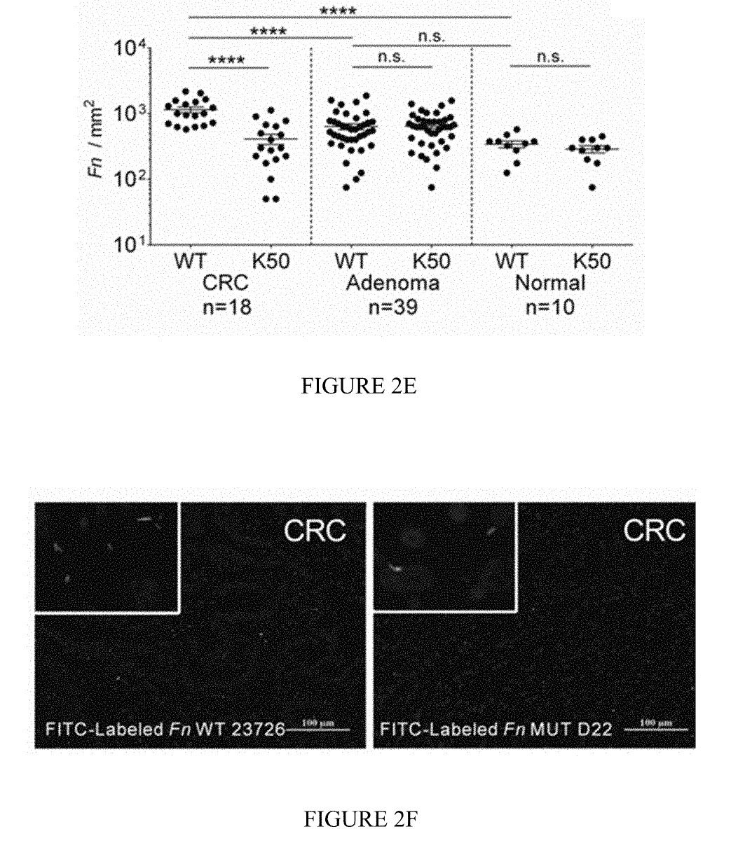

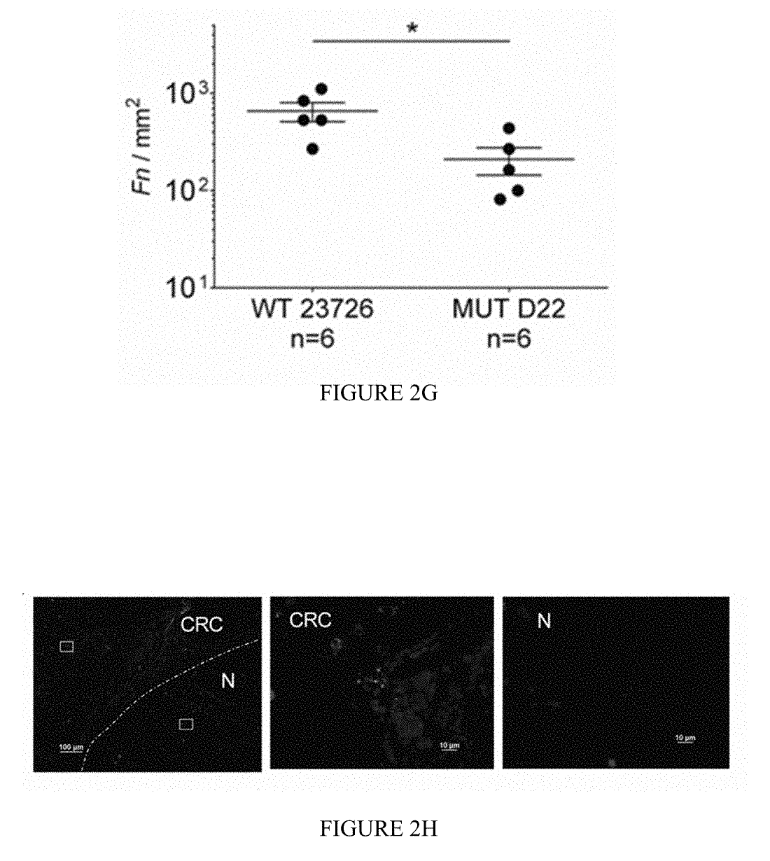

The Fap2 surface protein of F. nucleatum ATCC 23726 is a galactose-binding lectin that mediates fusobacterial hemagglutination. Fap2 was identified by screening a F. nucleatum ATCC 23726 transposon mutant library for clones unable to hemagglutinate. The selected non-hemagglutinating mutants K50 and D22 both harbored the transposon in their fap2 gene (Coppenhagen-Glazer et al., 2015; Infection and Immunity March 2015 Volume 83 Number 3). To test if Fap2 mediates binding of F. nucleatum ATCC 23726 to tumors that overexpress Gal-GalNAc, a hemagglutination assays in the presence or absence of GalNAc using WT Fn and two Fap2-inactivated mutants, K50 and D22 was performed (FIG. 2A). These hemagglutination data suggest that Fap2 mediates Gal-GalNAc binding by fusobacteria. FIGS. 2B-2C show that GalNAc inhibits binding of F. nucleatum ATCC 23726 to human CRC tissue sections. Both Fap2-inactivated mutants K50 and D22 display impaired attachment to human colon adenocarcinoma sections compared with the wild type F. nucleatum ATCC 23726 parental strain with a mean overall reduction in abundance of 2.8 and 3.1 fold, respectively (FIGS. 2D-2G). Similar to PNA binding (FIG. 1B), attachment of F. nucleatum ATCC 23726 to adenoma sections overall is not different from binding to normal colon tissues, nor is it different from K50 mutant binding (FIG. 2E). In addition, fluorescence microscopy analysis of human CRC sections demonstrates co-localization (81.6%) of FITC-labeled Fap2-expressing—F. nucleatum ATCC 23726 with tumor Gal-GalNAc detected in tumor sections from three individuals (visualized with Alexa Fluor® 647-conjugated PNA and FITC labeled F. nucleatum ATCC 23726) (FIG. 2H). These data support that F. nucleatum Fap2 and tumor-expressed Gal-GalNAc play an important role in F. nucleatum CRC enrichment and localization.

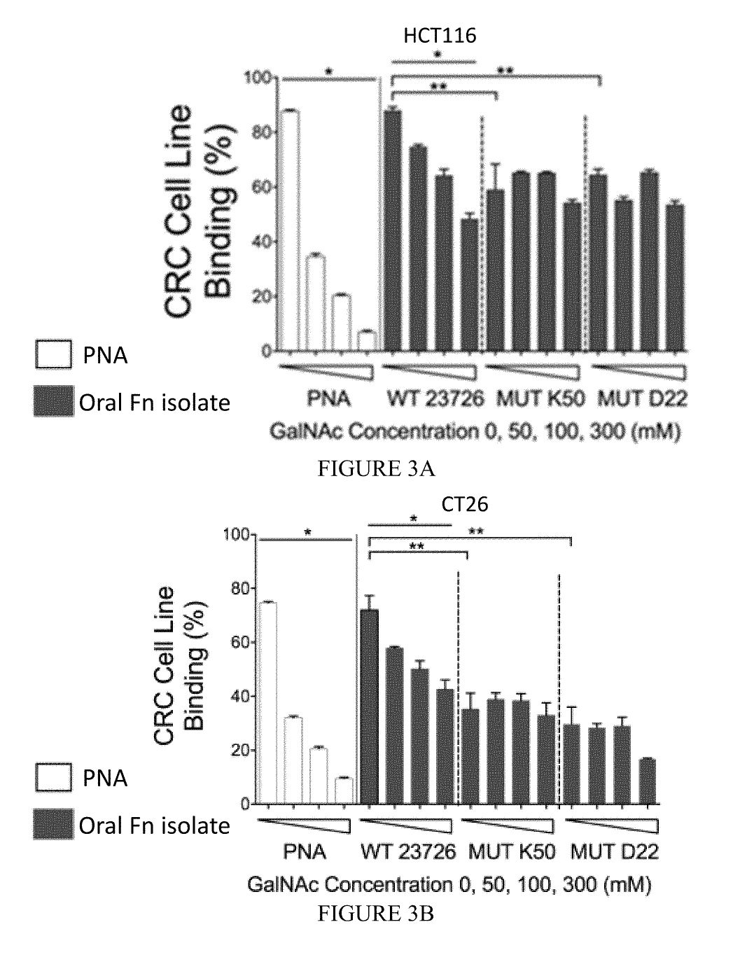

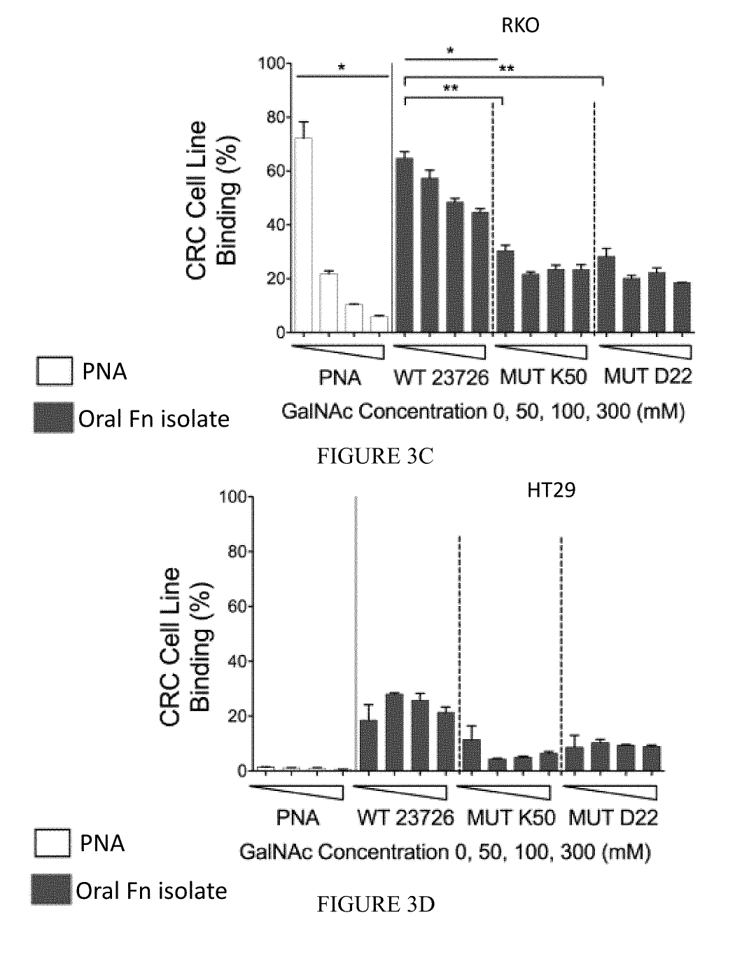

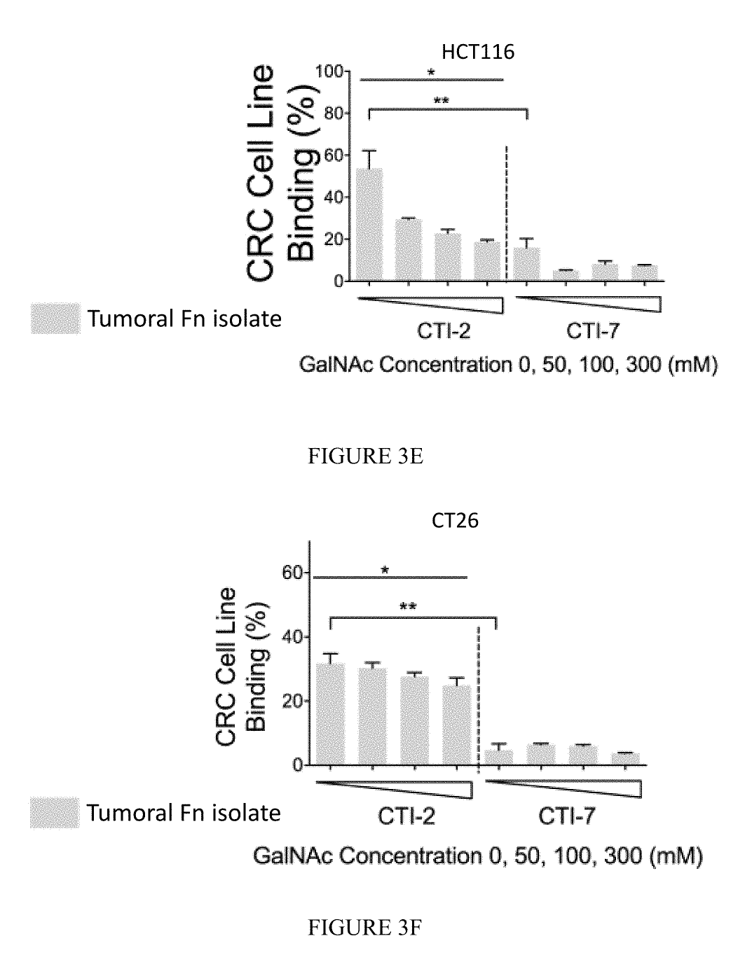

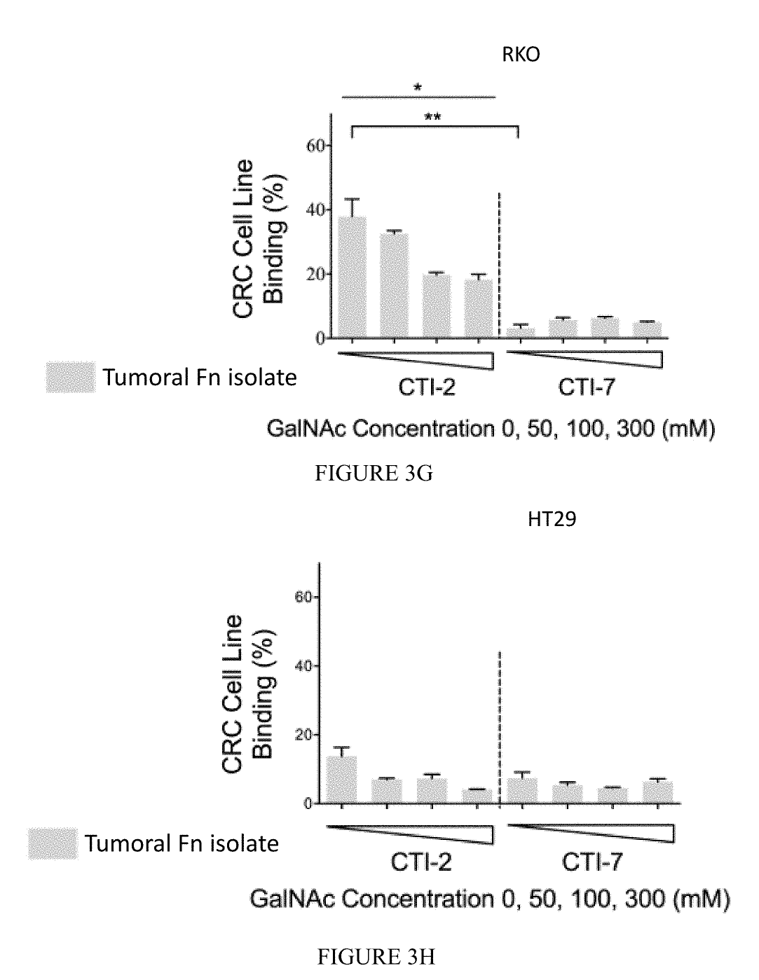

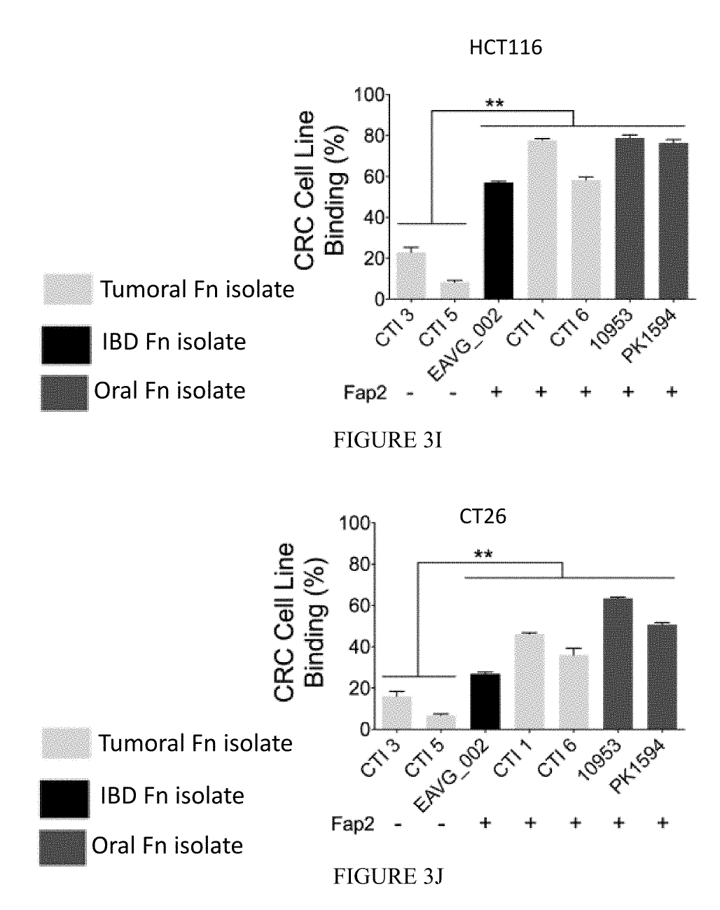

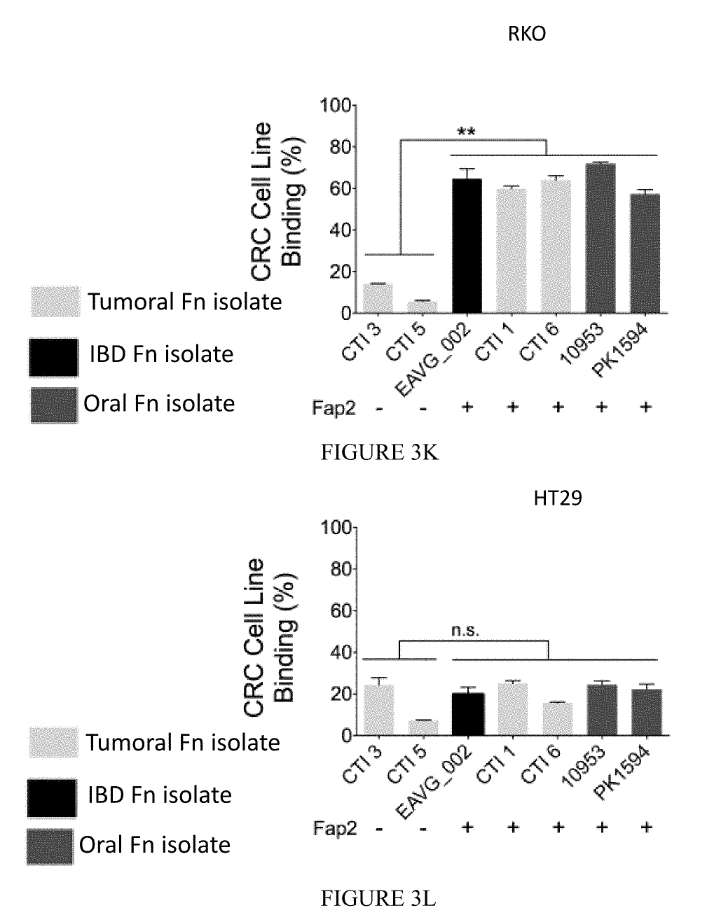

To confirm that fusobacterial attachment to CRC is Gal-GalNAc mediated, both flow cytometry and competition assays were employed. Flow cytometry analysis of the attachment of FITC-labeled F. nucleatum ATCC 23726 to human and mouse CRC cell lines revealed a correlation between bacterial attachment and cell line Gal-GalNAc expression levels measured using FITC-labeled PNA. Human HCT116 colon carcinoma cells, which expressed the highest amounts of Gal-GalNAc (mean 87.7% of cells binding PNA above threshold, FIGS. 3A-3D) bind the highest amounts of fusobacteria (mean 87.9% of cells binding above threshold, FIG. 3A). Mouse CT26 (FIG. 3B) and human RKO (FIG. 3C) CRC cells, expressing intermediate levels of Gal-GalNAc (means 74.6% and 72.1% respectively), bind intermediate amounts of F. nucleatum ATCC 23726 (means 71.8% and 64.8% respectively). Human HT29 CRC cells (FIG. 3D) that express low levels of Gal-GalNAc (mean 1.43%), demonstrate lower (mean 18.6%) fusobacterial attachment levels. Furthermore, binding of F. nucleatum ATCC 23726 to the high and intermediate Gal-GalNAc-expressing cell lines is inhibited by GalNAc in a statistically significant, dose-dependent manner (p=0.04167,FIGS. 3A-3D). These findings corroborate the importance of the Gal-GalNAc moiety for the attachment of the fusobacteria that was evaluated. In agreement with the results demonstrating the role of Fap2 in fusobacterial attachment to CRC sections (FIGS. 2D-2G), both Fap2-inactivated F. nucleatum ATCC 23726 mutants K50 and D22 have significantly impaired attachment to the high and intermediate Gal-GalNAc-expressing CRC cell lines, compared with the wild type parental strain (p<0.01, FIGS. 3A-3D). The residual binding of K50 and D22 to the CRC cells, is not GalNAc sensitive, confirming the role of Fap2 in Gal-GalNAc-mediated F. nucleatum ATCC 23726 CRC attachment. This residual binding may be FadA-mediated (Coppenhagen-Glazer et al., 2015, ibid; Han et al., 2005, Journal of bacteriology, Aug. 5330-5340). Binding of both Fap2 mutants to the HCT116 cells is higher than to the other tested cells suggesting that this cell line may express additional fusobacterial-binding ligands.

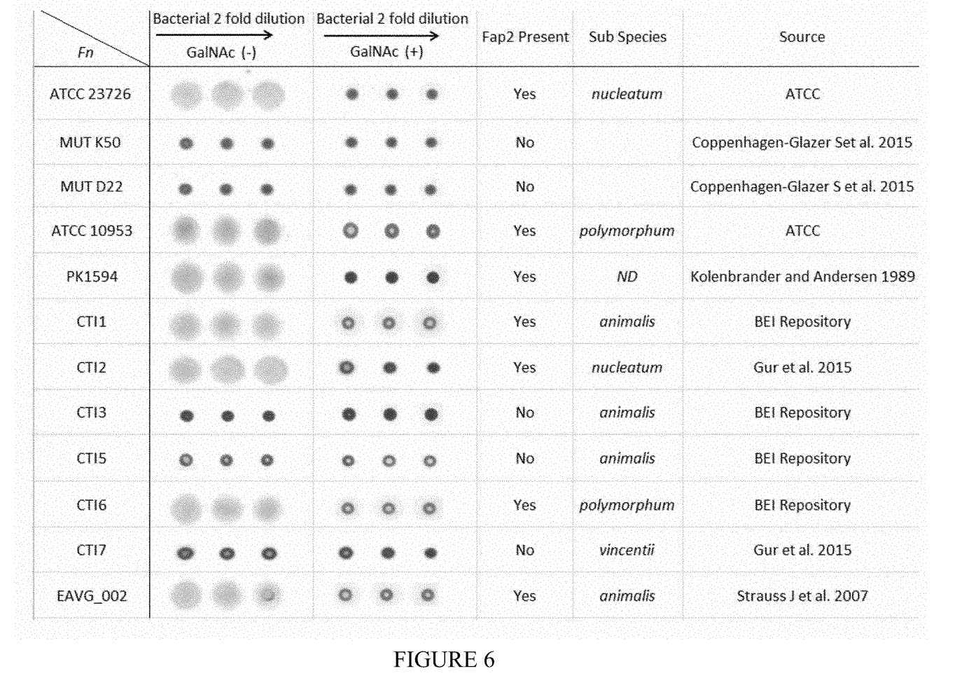

Next, Gal-GalNAc mediating CRC-binding by F. nucleatum strains CTI-2 and CTI-7, which were isolated from human CRC samples (Gur et al., 2015, ibid) was tested. While CTI-2 possesses the fap2 gene and its hemagglutination is inhibited by GalNAc, fap2 is not found in CTI-7's genome and CTI-7 does not hemagglutinate (FIG. 6). While both strains bind the low Gal-GalNAc-expressing HT-29 CRC cells in a similar manner, binding of CTI-2 to the high and intermediate Gal-GalNAc-expressing HCT116, CT26 and RKO CRC cells is significantly higher than that of the non-hemagglutinating, naturally Fap2-deficient CTI-7 (p<0.01, FIGS. 3E-3H). Binding of CTI-2 to the high and intermediate Gal-GalNAc-expressing CRC cells is inhibited by the addition of soluble GalNAc in a dose-dependent manner (p=0.04167, FIGS. 3E-3H), but binding of the Fap2-deficient CTI-7 is not (FIGS. 3E-3H). Correlation between Fap2 expression (detected by hemagglutination), (FIG. 6) and attachment to GalNAc-expressing CRC cell lines is also observed in 4 additional CRC F. nucleatum isolates, 2 F. nucleatum oral strains, and one F. nucleatum strain isolated from a patient with inflammatory bowel disease (FIG. 3I-3L).

### Example 3

### Bloodborne F. nucleatum Preferentially Colonizes Colorectal Tumors

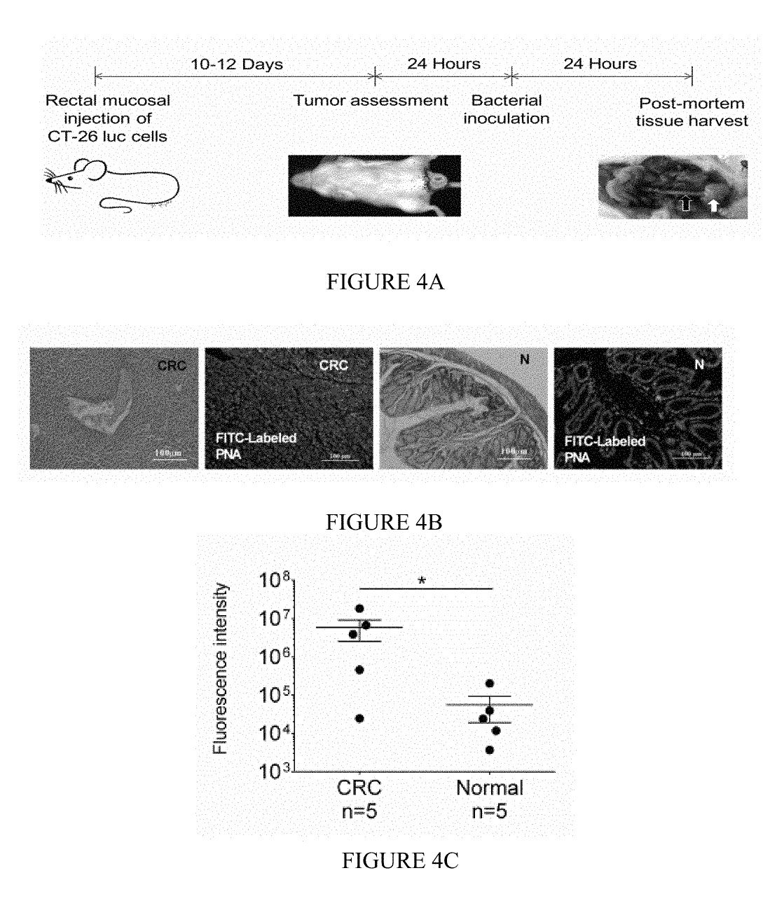

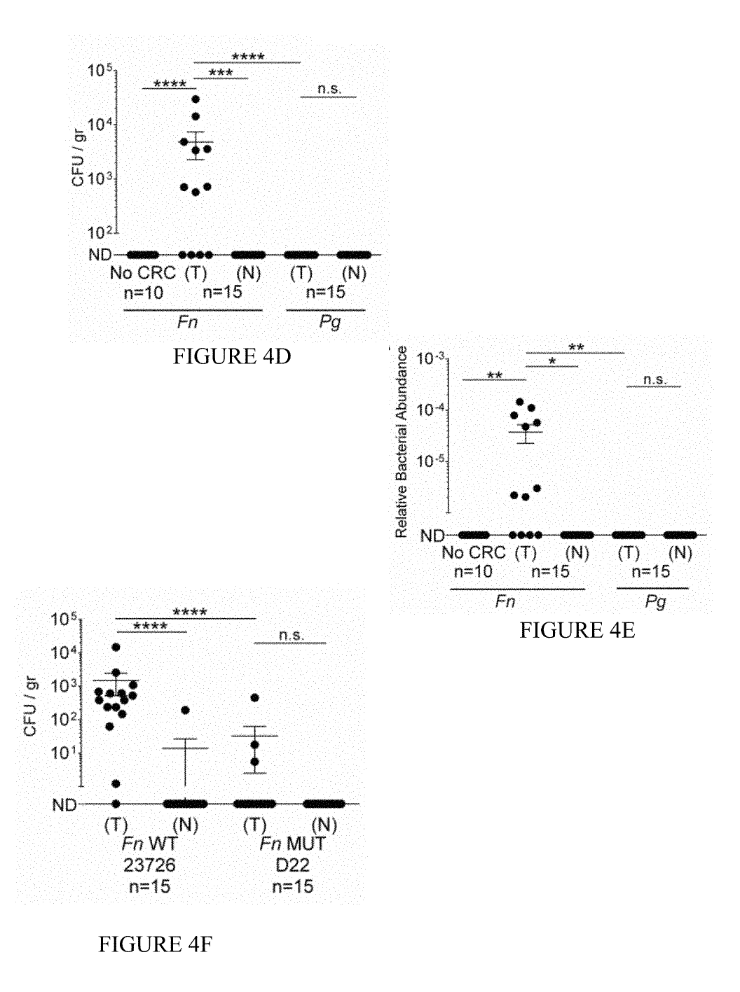

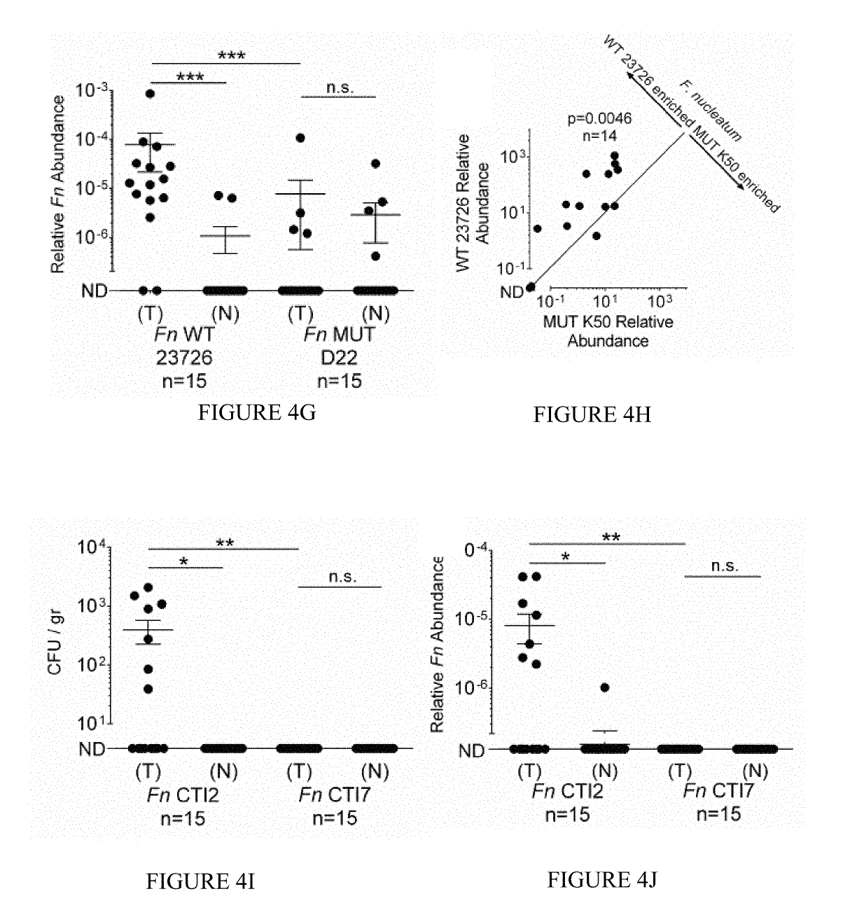

To test whether blood-borne fusobacteria can localize to CRC, the orthotopic rectal CT26 adenocarcinoma model described in Kolodkin-Gal et al. (2009, Gene Ther. 16, 905-915) was employed (FIG. 4A). CT26 cells stably transfected with the luciferase (luc) gene (CT26-luc) were injected under the mucosa of the distal rectum of BALB/cJ wild type mice and tumor volume and spread were assessed both by real-time imaging of luciferase expression and by direct measurement of rectal tumors. Once tumors reached 2500 mm³, mice were randomized to a control group or inoculated with 5×10⁶-1×10⁷ F. nucleatum ATCC 23726 by tail vein injection. Tumors and adjacent non-cancerous colon samples were harvested 24 hours post inoculation. Consistent with the samples from human colon adenocarcinoma, Gal-GalNAc (measured using FITC-labeled PNA) is overexpressed in the mouse CRC sections compared to sections prepared from adjacent normal colon tissues (FIGS. 4B-4C). In agreement with prior work in mouse models and humans (Kostic et al., 2013. ibid; Kostic et al., 2012, ibid), the abundance of fusobacteria in tumor tissues is significantly higher than in adjacent normal tissues both by plating and qPCR (p=0.0005 and p=0.0117 respectively, FIGS. 4D-4E). Also, intravenously inoculated fusobacteria are not found in the colons of control mice without CRC (FIGS. 4D-4E), suggesting that the presence of dysplastic or neoplastic lesions assists or is required for colonic localization of bloodborne fusobacteria. A tail vein injection of fusobacteria in Apc^{Min/+} mice was also performed. In these experiments, mice were injected after the 12^{th} week of age to ensure that the mice would have ample numbers of small intestinal adenomas. In the mouse facility used for the experiments, very rarely colonic adenomas of Apc^{Min/+} mice were observed without fusobacterial inoculation. Twenty-four hours after injection, wild type F. nucleatum ATCC 23726 were detected in small intestinal tissues from C57BL/6 Apc^{Min/+} mice by qPCR in 11/12 (91.7%) samples and 0/6 samples from C57BL/6 wild type mice. When C57BL/6 Apc^{Min/+} and wild type mice were injected with the K50, F. nucleatum was detected in 9/16 (56%) and 0/6 samples, respectively. Thus, colonization of the Fap2-expressing wild type strain (91.7%) is significantly higher than that of K50 9/16 (56%) (p=0.022 Mann-Whitney U test). These data indicate that Fap2 plays a role in F. nucleatum tumor enrichment in this model; however, the small intestinal localization of these tumors as well as the fact that Apc^{Min/30} adenoma histology does not fully recapitulate the spectrum of human colonic adenoma histology complicates interpretation and application to humans.

Tumor colonization does not appear to be a general feature of oral anaerobic bacteria associated with peridontitis. Porphyromonas gingivalis is an oral Gram-negative, anaerobic periodontal bacterium (Hajishengallis et al., 2011, Cell Host Microbe 10, 497-506) that was previously found to be overabundant in gingival squamous cell carcinoma (Katz et al., 2011, Int. J. Oral Sci. 3, 209-215.; Whitmore and Lamont, 2014, PLoS Pathog. 10, e1003933).

When mice were intravenously inoculated with P. gingivalis, its levels in tumors are below the limit of detection both by culturing (˜10 CFU/gr tissue) and qPCR (FIGS. 4D-4E). Thus, F. nucleatum likely harbors distinctive features that underpin its tumor localization, such as Fap2 the focus of this description and FadA (Rubinstein et al., 2013, ibid).

### Example 4

### Fap2 Mediates CRC Colonization by F. nucleatum in the CT26 Colorectal Cancer Model

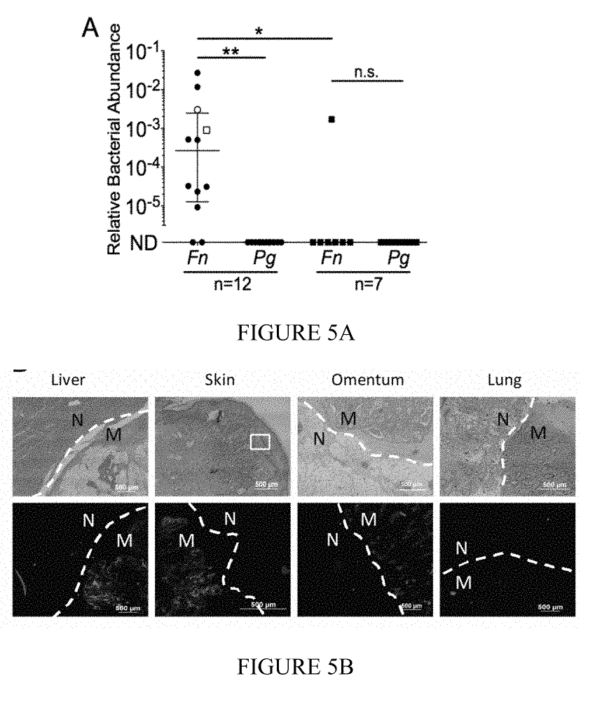

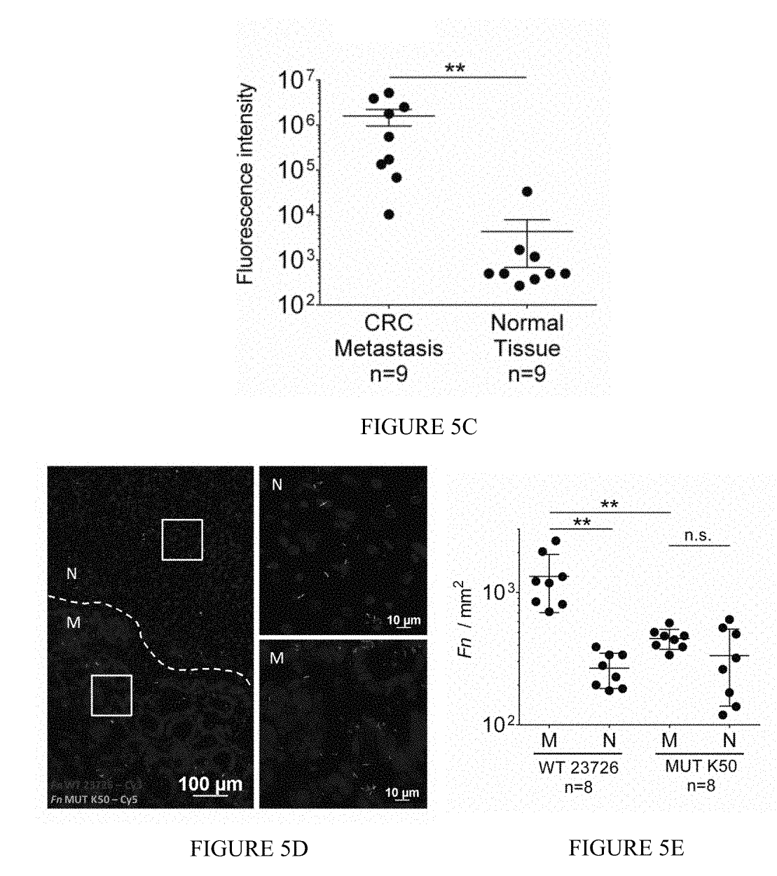

The orthotopic CT26 colorectal cancer model was also employed to evaluate the role of Fap2 in CRC localization by fusobacteria. Mice were inoculated with wild type (Fap2-expressing) F. nucleatum ATCC 23726 or with the Fap2-inactivated mutant D22. CRC colonization by the Fap2-deficient mutant D22 is significantly lower than that of the Fap2 sufficient ATCC 23726 parental strain as determined both by colony counting [45.6 fold less, (p<0.0001) FIG. 4F] and by qPCR [10.1 fold less, (p=0.0002) FIGS. 4F-4G]. Moreover, while CRC colonization by the Fap2 expressing F. nucleatum ATCC 23726 strain is significantly higher than that of the adjacent normal colon (p<0.0001, p=0.0008, FIG. 4F and G respectively), CRC colonization by D22 is not (FIG. 4F-4G). Co-challenge with ATCC 23726 and the other Fap2 mutant K50 co-injected into the CRC mouse model, confirm the involvement of Fap2 in CRC colonization by F. nucleatum ATCC 23726 (mean of competition index 25.5, p=0.0046; FIG. 4H). Using human colonic adenocarcinoma isolates, Fap2-expressing CTI-2 strain (FIG. 6) were found to be abundant in the tumors; however, the Fap2-deficient CTI-7 strain is not detected in the tumors by plating (FIG. 4I) and qPCR (FIG. 4J). These results imply that while neoplastic tissues play a critical role in fusobacterial tumor enrichment, fusobacterial CRC-specific enrichment is also Fap2 dependent. Next, fusobacteria ability to localize to CRC metastasis and whether this localization is Gal-GalNAc-Fap2 mediated were evaluated. F. nucleatum was detected in human CRC metastases by qPCR (FIG. 5A, 10/12 tested metastases), consistent with prior preliminary observations (Kostic et al. 2012, ibid); but fusobacteria were not detected in 6/7 samples taken from tumor-free liver biopsies (FIG. 5A). Presence of fusobacteria in CRC-metastasis colonization appears to be specific insofar as gDNA of P. gingivalis is not detected in the tested samples. Similar to primary colon adenocarcinoma, Gal-GalNAc is overexpressed (in comparison to adjacent normal tissue) in all of the tested metastases, from a variety of organs (FIGS. 5B-5C). As was observed in CRC primary tumors, ex vivo binding of F. nucleatum ATCC 23726 to CRC metastases sections was Fap2-dependent with reduced attachment of the Fap2—inactivated mutant K50 as compared to wild type (FIGS. 5D-5E).

### Example 5

### Gal-GalNAc Levels are Elevated in Adenocarcinomas

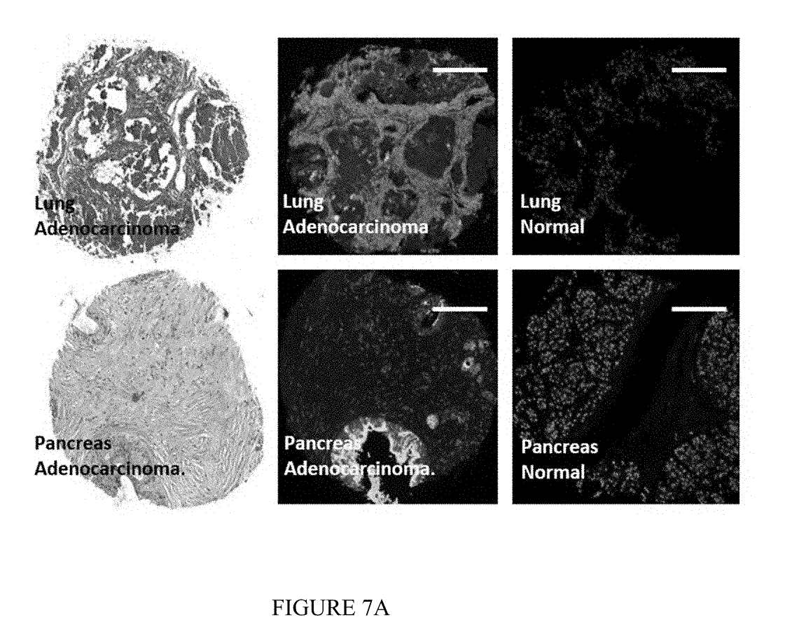

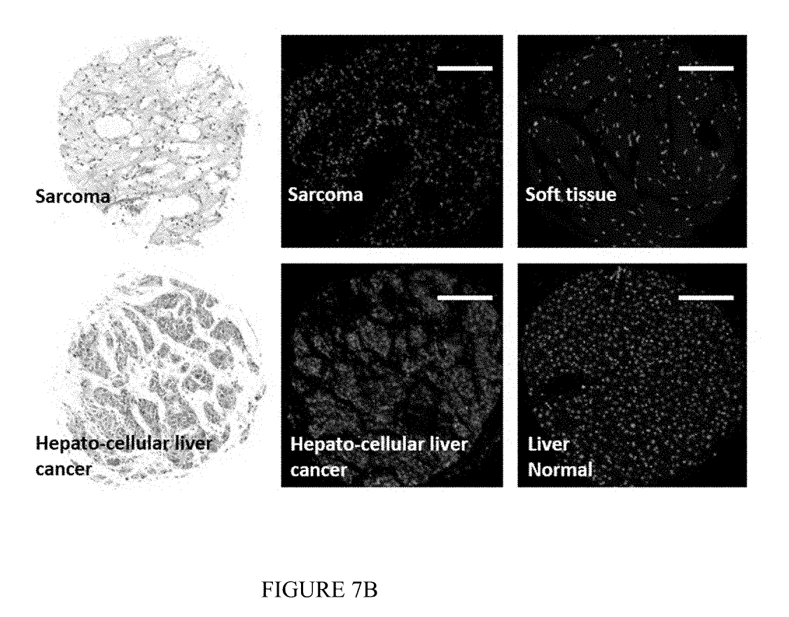

Tissue microarrays (TMAs) (Boimax inc. MC5003b, MC2082a, and BN1002b) that contain samples of 20 different tumors and matching normal tissue controls, were screened for Gal-GalNAc levels using a fluorescently labeled, Gal-GalNAc—specific, peanut agglutinin (PNA) lectin, as described in Abed et al., 2016, ibid. Representative images of sections of tumors that display high Gal-GalNAc levels (lung and pancreas adenocarcinomas) and of their matching controls (that display low Gal-GalNAc levels) can be seen in FIG. 7A. Images of representative tumors that display low Gal-GalNAc levels are presented in FIG. 7B.

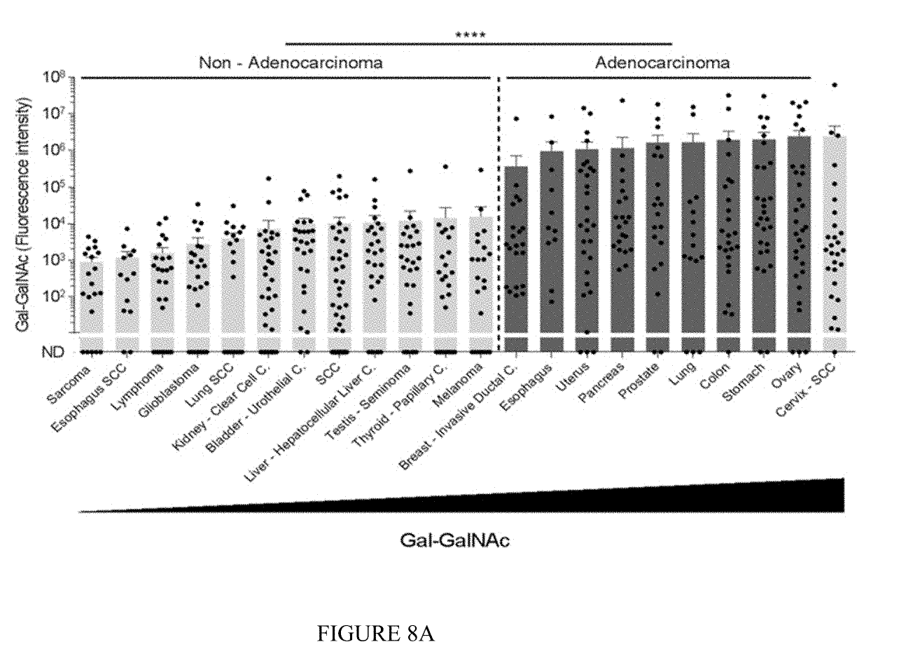

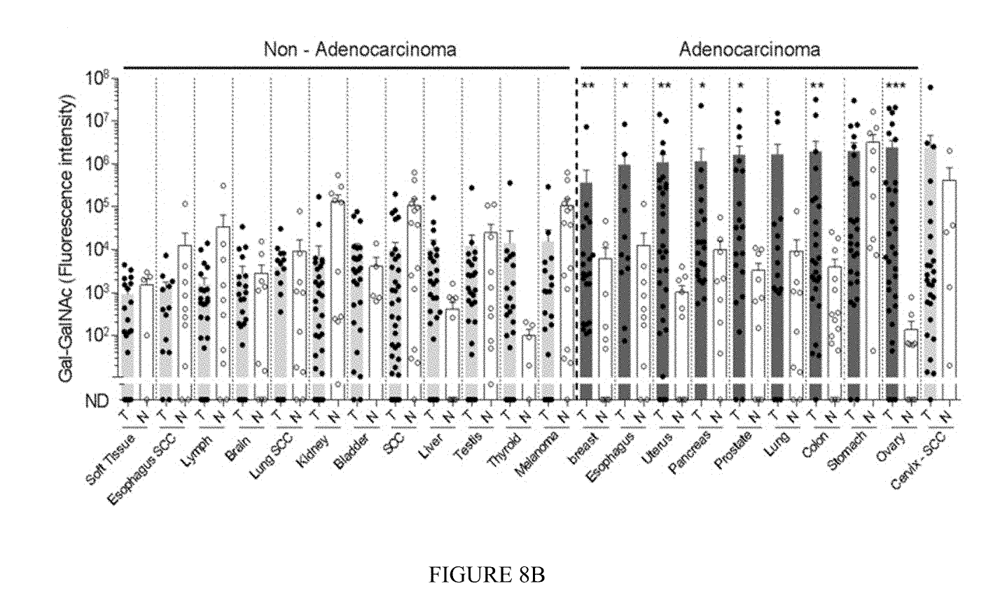

Next, the tested cancers were arranged according to their Gal-GalNAc levels (FIG. 8A). High Gal-GalNAc levels were detected in 10 tumors out of the 20 tested (FIG. 8A). These tumors were of epithelial tissue with glandular origin or/and glandular characteristics, 9 of them adenocarcinomas (of stomach, prostate, ovary, colon, uterus, pancreas, breast, lung and esophagus) and one a squamous cell carcinoma of the cervix. The Gal-GalNAc levels in 8 of these tumors, were higher than that in the matching normal tissue controls, 7 of them (all adenocarcinomas) with statistical significance (FIG. 8B). The Gal-GalNAc levels in the stomach and cervix normal control samples were high and similar to those in the respective cancers. Conversely, in the non-adenocarcinoma tumors, Gal-GalNAc levels were similar to those in the matching normal tissue controls (FIG. 8B).

The results above suggest that fusobacteria home-to and colonize Gal-GalNAc over-expressing cancers. As F. nucleatum was shown to accelerate tumor progression (Kostic et al., 2013 ibid; Rubinstein et al., 2013, ibid), fusobacterial elimination in these tumors should improve treatment outcome. In addition, Fap2 that was found to mediate F. nucleatum colonization to CRC, can be used as a targeting moiety for imaging or therapeutic agent toward cancer cells characterized by high levels of Gal-GalNAc.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation.

1. A composition comprising:
(i) a Fap2 protein or a fragment thereof comprising a Gal-GalNAc biding site; and
(ii) a therapeutic or diagnostic agent.

2. The composition of claim 1, wherein Fap2 is directly coupled to the therapeutic or diagnostic agent.

3. The composition of claim 1, said composition is selected from the group consisting of a liposome, a nanoparticle or a micro particle.

4. The composition of claim 1, wherein the Fap2 protein is a mutated protein.

5. The composition of claim 1, wherein the composition is formulated for parenteral administration.

6. The composition of claim 1, wherein the diagnostic agent is selected from the group consisting of: fluorescent agent, radio-imaging agent, photo-imaging agent, and an agent used to perform or enhance CT, MRI, or ultrasound.

7. The composition of claim 1, wherein the composition is a pharmaceutical composition further comprising an acceptable pharmaceutical carrier.

8. The composition of claim 7, wherein the therapeutic agent is a chemotherapeutic agent.

9. A method of treating cancer characterized by elevated amounts of Gal-GalNAc in a subject, the method comprising administering to the subject a therapeutically effective amount of the pharmaceutical composition of claim 7.

10. The method of claim 9, wherein the cancer is adenocarcinoma.

11. The method of claim 9, wherein the cancer is colorectal cancer (CRC).

12. A method of diagnosing cancer in a subject, said cancer is characterized by elevated amounts of Gal-GalNAc molecules on cell surface, the method comprising administering to the subject the composition according to claim 1.

13. A method of diagnosing cancer in a subject, said cancer is characterized by elevated amounts of Gal-GalNAc molecules on cell surface, the method comprising determining the expression level of Gal-GalNAc in a biological sample of said subject using a composition according to claim 1 and comparing the expression level of Gal-GalNAc with a control or a reference sample.

14. A method of treating cancer characterized by elevated amounts of Gal-GalNAc, the method comprising administering to a subject in need thereof a therapeutically effective amount of at least one agent that reduces the binding of Fap2 to Gal-GalNAc present on cancer cells.

15. The method of claim 14, wherein the cancer is adenocarcinoma.

16. The method of claim 14, wherein the cancer is CRC.

17. The method of claim 14, wherein the agent is selected from the group consisting of: antibody, polypeptide, siRNA, and small molecule.

18. The method of claim 14, wherein the antibody is against Fap2 or Gal-GalNAc.

19. The method of claim 14, wherein the agent is a mutated Fap2.

20. The method of claim 14, wherein the agent is administered in conjunction with one or more chemotherapeutic agents, immunotherapeutic agents, surgery or radiotherapy.
